(19) United States
(12) Patent Application Publication
(10) Pub. No.: US 2006/0194969 A1
(43) Pub. Date: Aug. 31, 2006

(54) 4-Substituted benzimidazoles and their uses as inhibitors of gastric secretion

(76) Inventors: Peter Jan Zimmermann, Radolfzell (DE); Wilm Buhr, Konstanz (DE)

Correspondence Address: NATH & ASSOCIATES PLLC, 112 South West Street, Alexandria, VA, 22314, US

(21) Appl. No.: 10/537,693

(22) PCT Filed: Dec. 11, 2003
(86) PCT No.: PCT/EP03/14069

§ 371 Date: Mar. 13, 2006

(30) Foreign Application Priority Data
Dec. 13, 2002 (EP) 02027874.3
Sep. 20, 2003 (EP) 03021348.2

(51) Int. Cl. C07D 413/02; C07D 403/02

(52) U.S. Cl. 544/139; 548/304.1; 544/366; 548/305.4; 546/199

(57) ABSTRACT

The invention relates to 6-substituted benzimidazoles of formula 1, 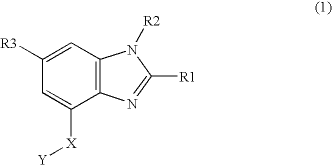 in which the substituents and symbols have the meanings indicated in the description. The compounds have gastric secretion inhibiting and excellent gastric and intestinal protective action properties.

### TECHNICAL FIELD

The invention relates to novel compounds, which are used in the pharmaceutical industry as active compounds for the production of medicaments.

### PRIOR ART

In the European patent application 266326 (which corresponds to U.S. Pat. No. 5,106,862), benzimidazole derivatives having a very broad variety of substituents are disclosed, which are said to be active as antiulcer agents. The international patent application WO 97/47603 (which corresponds to U.S. Pat. No. 6,465,505) discloses benzimidazole derivatives substituted by a 2,6-dialkyl phenyl moiety, which are effective as inhibitors of the H⁺, K⁺-ATPase.

### SUMMARY OF THE INVENTION

The invention relates to compounds of the formula 1 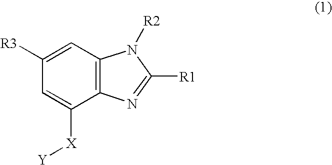
in which
R1 is hydrogen, 1-4C-alkyl, 3-7C-cycloalkyl, 3-7C-cycloalkyl-1-4C-alkyl, 1-4C-alkoxy, 1-4C-alkoxy- 1-4C-alkyl, 1-4C-alkoxycarbonyl, 2-4C-alkenyl, 2-4C-alkynyl, fluoro-1-4C-alkyl, hydroxy-1-4C-alkyl, mono- or di-1-4C-alkylamino or 1-4C-alkylcarbonyloxy-1-4C-alkyl
R2 is hydrogen, 1-4C-alkyl, aryl, 3-7C-cycloalkyl, 3-7C-cycloalkyl-1-4C-alkyl, 1-4C-alkoxycarbonyl, mono- or di-1-4C-alkylamino 1-4C-alkylcarbonyl, hydroxy-1-4C-alkyl, fluoro-2-4C-alkyl, aryl-1-4C-alkoxy-1-4C-alkyl, hydroxy or 1-4C-alkoxy
R3 is hydrogen, halogen, fluoro-1-4C-alkyl, carboxyl, —CO-1-4C-alkoxy, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, cyano, the group —CO—NR31R32, the group SO₂—NR31R32 or the group Het,
   where
   R31 is hydrogen, hydroxyl, 1-7C-alkyl, hydroxy-1-4C-alkyl, 1-4C-alkoxy-1-4C-alkyl or 3-7C-cycloalkyl, amino and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group and
   Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol and tetrazol
   where
   R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R34 is hydrogen, 1-4C-alkyl, 1-4C-alkoxy, 1-4C-alkoxycarbonyl, halogen, trifluoromethyl or hydroxy,
   R35 is hydrogen, 1-4C-alkyl, 1-4C-alkoxy, 1-4C-alkoxycarbonyl, halogen, trifluoromethyl or hydroxy,
X is O (oxygen) or NH and
Y has either the meaning —CH₂—Ar
   wherein
   Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   or Y denotes the group gp 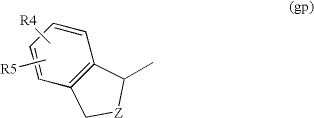
   wherein
   Z has the meaning —CHR8- or —CHR8-CHR9-
   where in, Ar and/or in the group gp
   R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxy,
   R6 is hydrogen, 1-4C-alkyl or halogen and
   R7 is hydrogen, 1-4C-alkyl or halogen,
   R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy
   R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy,
   and wherein
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      with the proviso that R3 does not have the meaning hydrogen or halogen when Y denotes —CH₂—Ar and R2 denotes hydrogen, 1-4C-alkyl or 3-7C-cycloalkyl-1-4C-alkyl,
      and the salts of these compounds.

1-4C-Alkyl represents straight-chain or branched alkyl groups having 1 to 4 carbon atoms. Examples which may be mentioned are the butyl, isobutyl, sec-butyl, tert-butyl, propyl, isopropyl, ethyl and the methyl group.

3-7C-Cycloalkyl represents cyclopropyl, cyclobutyl, cyclopentyl, cyclohexyl and cycloheptyl, of which cyclopropyl, cyclobutyl and cyclopentyl are preferred.

3-7C-Cycloalkyl-1-4C-alkyl represents one of the aforementioned 1-4C-alkyl groups, which is substituted by one of the aforementioned 3-7C-cycloalkyl groups. Examples which may be mentioned are the cyclopropylmethyl, the cyclohexylmethyl and the cyclohexylethyl group.

1-4C-Alkoxy represents groups, which in addition to the oxygen atom contain a straight-chain or branched alkyl group having 1 to 4 carbon atoms. Examples which may be mentioned are the butoxy, isobutoxy, sec-butoxy, tert-butoxy, propoxy, isopropoxy and preferably the ethoxy and methoxy group.

1-4C-Alkoxy-1-4C-alkyl represents one of the aforementioned 1-4C-alkyl groups, which is substituted by one of the aforementioned 1-4C-alkoxy groups. Examples which may be mentioned are the methoxy-methyl, the methoxyethyl group and the butoxyethyl group.

1-4C-Alkoxycarbonyl (—CO-1-4C-alkoxy) represents a carbonyl group, to which one of the aforementioned 1-4C-alkoxy groups is bonded. Examples which may be mentioned are the methoxycarbonyl (CH₃O—C(O)—) and the ethoxycarbonyl group (CH₃CH₂O—C(O)—).

2-4C-Alkenyl represents straight-chain or branched alkenyl groups having 2 to 4 carbon atoms. Examples which may be mentioned are the 2-butenyl, 3-butenyl, 1-propenyl and the 2-propenyl group (allyl group).

2-4C-Alkynyl represents straight-chain or branched alkynyl groups having 2 to 4 carbon atoms. Examples which may be mentioned are the 2-butynyl, 3-butynyl, and preferably the 2-propynyl, group (propargyl group).

Fluoro-1-4C-alkyl represents one of the aforementioned 1-C-alkyl groups, which is substituted by one or more fluorine atoms. An example which may be mentioned is the trifluoromethyl group.

Hydroxy-1-4C-alkyl represents aforementioned 1-4C-alkyl groups, which are substituted by a hydroxy group. Examples which may be mentioned are the hydroxymethyl, the 2-hydroxyethyl and the 3-hydroxypropyl group.

1-4C-Alkylcarbonyl represents a group, which in addition to the carbonyl group contains one of the aforementioned 1-4C-alkyl groups. An example which may be mentioned is the acetyl group.

Mono- or di-1-4C-alkylamino represents an amino group, which is substituted by one or by two—identical or different—groups from the aforementioned 1-4C-alkyl groups. Examples which may be mentioned are the dimethylamino, the diethylamino and the diisopropylamino group.

Mono- or di-1-4C-alkylamino-1-4C-alkylcarbonyl represents a 1-4C-alkylcarbonyl group, which is substituted by a mono- or di-1-4C-alkylamino groups. Examples, which may be mentioned, are the dimethylamino-methylcarbonyl and the dimethylamino-ethylcarbonyl group.

Fluoro-2-4C-alkyl represents a 2-4C-alkyl groups, which is substituted by one or more fluorine atoms. An example which may be mentioned is the 2,2,2-trifluoroethyl group.

Aryl-1-4C-alkoxy denotes an aryl-substituted 1-4C-alkoxy radical. An example which may be mentioned is the benzyloxy radical.

Aryl-1-4C-alkoxy-1-4C-alkyl denotes one of the aforementioned 1-4C-alkyl groups, which is substituted by one of the aforementioned aryl-1-4C-alkoxy radicals. An example which may be mentioned is the benzyloxymethyl radical.

Halogen within the meaning of the invention is bromo, chloro and fluoro.

1-C-Alkoxy-1-4C-alkoxy represents one of the aforementioned 1-4C-alkoxy groups, which is substituted by a further 1-4C-alkoxy group. Examples which may be mentioned are the groups 2-methoxy)ethoxy (CH₃—O—CH₂—CH₂—O—) and 2-(ethoxy)ethoxy (CH₃—CH₂—O—CH₂—CH₂—O—).

1-4C-Alkoxy-1-4C-alkoxy-1-4C-alkyl represents one of the aforementioned 1-4C-alkoxy-1-4C-alkyl groups, which is substituted by one of the aforementioned 1-4C-alkoxy groups. An example which may be mentioned is the group 2-(methoxy)ethoxymethyl (CH₃—O—CH₂—CH₂—O—CH₂—).

Fluoro-1-4C-alkoxy-1-4C-alkyl represents one of the aforementioned 1-4C-alkyl groups, which is substituted by a fluoro-1-4C-alkoxy group. Fluoro-1-4C-alkoxy in this case represents one of the aforementioned 1-4C-alkoxy groups, which is completely or mainly substituted by fluorine. Examples of completely or mainly fluoro-substituted 1-4C-alkoxy groups which may be mentioned are the 1,1,1,3,3,3-hexafluoro-2-propoxy, the 2-trifluoromethyl-2-propoxy, the 1,1,1-trifluoro-2-propoxy, the perfluoro-tert-butoxy, the 2,2,3,3,4,4,4-heptafluoro-1-butoxy, the 4,4,4-trifluoro-1-butoxy, the 2,2,3,3,3-pentafluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy group.

1-7C-Alkyl represents straight-chain or branched alkyl groups having 1 to 7 carbon atoms. Examples which may be mentioned are the heptyl, isoheptyl (5-methylhexyl), hexyl, isohexyl (4-methylpentyl), neo-hexyl (3,3-dimethylbutyl), pentyl, isopentyl (3-methylbutyl), neopentyl (2,2-dimethylpropyl), butyl, isobutyl, sec-butyl, tert-butyl, propyl, isopropyl, ethyl and the methyl group.

2-4C-Alkenyloxy represents groups, which in addition to the oxygen atom contain one of the abovementioned 2-4C-alkenyl groups. Examples, which may be mentioned, are the 2-butenyloxy, 3-butenyloxy, 1-propenyloxy and the 2-propenyloxy group (allyloxy group).

Carboxy-1-4C-alkyl represents 1-4C-alkyl groups which are substituted by a carboxyl group. Examples, which may be mentioned, are the carboxymethyl and the 2-carboxyethyl group.

1-4C-Alkoxycarbonyl-1-4C-alkyl represents 1-4C-alkyl groups, which are substituted by one of the abovementioned 1-4C-alkoxycarbonyl groups. Examples, which may be mentioned, are the Methoxycarbonylmethyl and the ethoxycarbonylmethyl group.

Aryl-1-4C-alkyl denotes an aryl-substituted 1-4C-alkyl radical. An example which may be mentioned is the benzyl radical.

1-4C-Alkylcarbonylamino represents an amino group to which a 1-4C-alkylcarbonyl group is bonded. Examples which may be mentioned are the propionylamino (C₃H₇C(O)NH—) and the acetylamino group (acetamido group) (CH₃C(O)NH—).

1-4C-Alkoxycarbonylamino represents an amino group, which is substituted by one of the aforementioned 1-4C-alkoxycarbonyl groups. Examples, which may be mentioned, are the ethoxycarbonylamino and the methoxycarbonylamino group.

1-4C-Alkoxy-1-4C-alkoxycarbonyl represents a carbonyl group, to which one of the aforementioned 1-4C-alkoxy-1-4C-alkoxy groups is bonded. Examples which may be mentioned are the 2-methoxy)-ethoxycarbonyl (CH₃—O—CH₂CH₂—O—CO—) and the 2-(ethoxy)ethoxycarbonyl group (CH₃CH₂—O—CH₂CH₂—O—CO—).

1-4C-Alkoxy-1-4C-alkoxycarbonylamino represents an amino group, which is substituted by one of the aforementioned 1-C-alkoxy-1-4C-alkoxycarbonyl groups. Examples which may be mentioned are the 2-(methoxy)ethoxycarbonylamino and the 2-(ethoxy)ethoxycarbonylamino group.

2-7C-Alkenyl represents straight-chain or branched alkenyl groups having 2 to 7 carbon atoms. Examples which may be mentioned are the 2-butenyl, 3-butenyl, 1-propenyl, the 2-propenyl (allyl) and the vinyl group. The aforementioned 2-4C-alkenyl groups are preferred.

2-7C-Alkenyl represents straight-chain or branched alkenyl groups having 2 to 7 carbon atoms. Examples which may be mentioned are the 2-butenyl, 3-butenyl, 1-propenyl, the 2-propenyl (allyl) and the vinyl group. The aforementioned 2-4C-alkenyl groups are preferred.

Oxo-substituted 1-4C-alkoxy represents a 1-4C-alkoxy group, which instead of a methylene group contains a carbonyl group. An example which may be mentioned is the 2-oxopropoxy group.

3-7C-Cycloalkoxy represents cyclopropyloxy, cyclobutyloxy, cyclopentyloxy, cyclohexyloxy and cycloheptyloxy, of which cyclopropyloxy, cyclobutyloxy and cyclopentyloxy are preferred.

3-7C-Cycloalkyl-1-4C-alkoxy represents one of the aforementioned 1-4C-alkoxy groups, which is substituted by one of the aforementioned 3-7C-cycloalkyl groups. Examples which may be mentioned are the cyclopropylmethoxy, the cyclobutylmethoxy and the cyclohexylethoxy group.

Hydroxy-1-4C-alkoxy represents aforementioned 1-4C-alkoxy groups, which are substituted by a hydroxy group. A preferred example which may be mentioned is the 2-hydroxyethoxy group.

1-4C-Alkoxy-1-4C-alkoxy-1-4C-alkoxy represents one of the aforementioned 1-4C-alkoxy groups, which is substituted by one of the aforementioned 1-4C-alkoxy-1-4C-alkoxy groups. A preferred example which may be mentioned is the methoxyethoxyethoxy group.

3-7C-Cycloalkoxy-1-4C-alkoxy represents one of the aforementioned 1-4C-alkoxy groups, which is substituted by one of the aforementioned 3-7C-cycloalkoxy groups. Examples which may be mentioned are the cyclopropoxymethoxy, the cyclobutoxymethoxy and the cyclohexyloxyethoxy group.

3-7C-Cycloalkyl-1-4C-alkoxy-1-4C-alkoxy represents one of the aforementioned 1-4C-alkoxy groups, which is substituted by one of the aforementioned 3-7C-cycloalkyl-1-4C-alkoxy groups. Examples which may be mentioned are the cyclopropylmethoxyethoxy, the cyclobutylmethoxyethoxy and the cyclohexylethoxyethoxy group.

1-4C-Alkylcarbonyloxy represents a 1-4C-alkylcarbonyl group which is bonded to an oxygen atom. An example which may be mentioned is the acetoxy group (CH₃CO—O—).

1-4C-Alkylcarbonyloxy-1-4C-alkyl represents one of the aforementioned 1-4C-alkyl groups, which is substituted by one of the aforementioned 1-4C-alkylcarbonyloxy groups. An example which may be mentioned is the acetoxymethyl group (CH₃CO—O—CH₂).

Halo-1-4C-alkoxy represents 1-4C-alkoxy groups which are completely or mainly substituted by halogen. “Mainly” in this connection means that more than half of the hydrogen atoms in the 1-4C-alkoxy groups are replaced by halogen atoms. Halo-1-4C-alkoxy groups are primarily chloro- and/or in particular fluoro-substituted 1-4C-alkoxy groups. Examples of halogen-substituted 1-4C-alkoxy groups which may be mentioned are the 2,2,2-trichloroethoxy, the hexachloroisopropoxy, the pentachloroisopropoxy, the 1,1,1-trichloro-3,3,3-trifluoro-2-propoxy, the 1,1,1-trichloro-2-methyl-2-propoxy, the 1,1,1-trichloro-2-propoxy, the 3-bromo-1,1,1-trifluoro-2-propoxy, the 3-bromo-1,1,1-trifluoro-2-butoxy, the 4-bromo-3,3,4,4-tetrafluoro-1-butoxy, the chlorodifluoromethoxy, the 1,1,1,3,3,3-hexafluoro-2-propoxy, the 2-trifluoromethyl-2-propoxy, the 1,1,1-trifluoro-2-propoxy, the perfluoro-tert-butoxy, the 2,2,3,3,4,4,4-heptafluoro-1-butoxy, the 4,4,4-trifluoro-1-butoxy, the 2,2,3,3,3-pentafluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy group.

Mono- or di-1-4C-alkylamino-1-4C-alkylcarbonyloxy represents a 1-4C-alkylcarbonyloxy group, which is substituted by one of the aforementioned mono- or di-1-C-alkylamino groups. Examples, which may be mentioned, are the dimethylamino-methylcarbonyloxy and the dimethylamino-ethylcarbonyloxy group.

1-4C-Alkoxy-1-4C-alkylcarbonyloxy represents one of the aforementioned 1-4C-alkylcarbonyloxy radicals which is substituted by one of the aforementioned 1-4C-alkoxy groups. An example, which may be mentioned, is the methoxymethylcarbonyloxy group.

Possible salts of compounds of the formula 1 or 2—depending on substitution—are especially all acid addition salts. Particular mention may be made of the pharmacologically tolerable salts of the inorganic and organic acids customarily used in pharmacy. Those suitable are water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, where the acids are used in salt preparation—depending on whether a mono- or polybasic acid is concerned and on which salt is desired—in an equimolar quantitative ratio or one differing therefrom.

Pharmacologically intolerable salts, which can initially be obtained, for example, as process products in the production of the compounds according to the invention on the industrial scale, are converted into the pharmacologically tolerable salts by processes known to the person skilled in the art

It is known to the person skilled in the art that the compounds according to invention and their salts, if, for example, they are isolated in crystalline form, can contain various amounts of solvents. The invention therefore also comprises all solvates and in particular all hydrates of the compounds of the formula I, and also all solvates and in particular all hydrates of the salts of the compounds of the formula I.

One embodiment (embodiment a) comprises compounds of the formula 1, in which

R1 is mono- or di-1-4C-alkylamino

R2, R3, X and Y have the meanings given above in the summary of the invention with the proviso that R3 does not have the meaning hydrogen or halogen when Y denotes —CH₂—Ar and

R2 denotes hydrogen, 1-4C-alkyl or 3-7C-cycloalkyl-1-4C-alkyl,

and the salts of these compounds.

One embodiment (embodiment b) comprises compounds of the formula 1, in which

R1 is 1-4C-alkylcarbonyloxy-1-4C-alkyl and

R2, R3, X and Y have the meanings given above in the summary of the invention

and the salts of these compounds.

Another embodiment (embodiment c) comprises compounds of the formula 1, in which

R2 is hydroxy or 1-4C-alkoxy and

R1, R3, X and Y have the meanings given above in the summary of the invention

and the salts of these compounds.

Another embodiment (embodiment d) comprises compounds of the formula 1, in which
R3 is cyano, the group SO₂—NR31R32 or the group Het and
   where
   R31 is hydrogen, hydroxyl, 1-7C-alkyl, hydroxy-1-4C-alkyl, 1-4C-alkoxy-1-4C-alkyl or 3-7C-cycloalkyl, amino and
   R32 is hydrogen, 1-7C-alkyl, hydroxy-1-C-alkyl or 1-4C-alkoxy-1-C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group and
   Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol and tetrazol
   where
   R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R34 is hydrogen, 1-4C-alkyl, 1-4C-alkoxy, 1-4C-alkoxycarbonyl, halogen, trifluoromethyl or hydroxy,
   R35 is hydrogen, 1-4C-alkyl, 1-4C-alkoxy, 1-4C-alkoxycarbonyl, halogen, trifluoromethyl or hydroxy,
      where
R1, R2, X and Y have the meanings given above in the summary of the invention
   and the salts of these compounds.

Another embodiment (embodiment e) comprises compounds of the formula 1, in which
R1 is hydrogen, 1-4C-alkyl, 3-7C-cycloalkyl, 3-7C-cycloalkyl-1-4C-alkyl, 1-4C-alkoxy, 1-4C-alkoxy- 1-4C-alkyl, 1-4C-alkoxycarbonyl, 2-4C-alkenyl, 2-4C-alkynyl, fluoro-1-4C-alkyl or hydroxy-1-4C-alkyl,
R2 is hydrogen, 1-4C-alkyl, aryl, 3-7C-cycloalkyl, 3-7C-cycloalkyl-1-4C-alkyl, 1-4C-alkoxycarbonyl, mono- or di-1-4C-alkylamino1-4C-alkylcarbonyl, hydroxy-1-4C-alkyl, fluoro-2-4C-alkyl, aryl-1-4C-alkoxy-1-4C-alkyl
R3 is hydrogen, halogen, fluoro-1-4C-alkyl, carboxyl, —CO-1-4C-alkoxy, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the group —CO—NR31R32,
   where
   R31 is hydrogen, hydroxyl, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
X is O (oxygen) or NH and
Y has either the meaning —CH₂—Ar
   wherein
   Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   or Y denotes the group gp 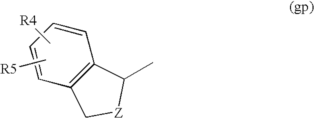
   wherein
   Z has the meaning —CHR8- or —CHR8-CHR9-
   where in Ar and/or in the group gp
   R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxy,
   R6 is hydrogen, 1-4C-alkyl or halogen and
   R7 is hydrogen, 1-4C-alkyl or halogen,
   R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy
   R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy,
   and wherein
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      with the proviso that R3 does not have the meaning hydrogen or halogen when Y denotes —CH₂—Ar, and the salts of these compounds.

Another embodiment (embodiment f) comprises compounds of the formula 1, in which
R1 is hydrogen, 1-4C-alkyl, 3-7C-cycloalkyl, 3-7C-cycloalkyl-1-4C-alkyl, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is hydrogen, 1-4C-alkyl, aryl, 3-7C-cycloalkyl, 3-7C-cycloalkyl-1-4C-alkyl, 1-4C-alkoxycarbonyl, mono- or di-1-4C-alkylamino1-4C-alkylcarbonyl, hydroxy-1-4C-alkyl or fluoro-2-4C-alkyl,
R3 is hydrogen, halogen, fluoro-1-4C-alkyl, carboxyl, —CO-1-4C-alkoxy, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the group —CO—NR31R32,
   where
   R31 is hydrogen, hydroxyl, 1-7C-alkyl, hydroxy-1-4C-alkyl or 1-4C-alkoxy-1-4C-alkyl
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino or morpholino group,
X is O (oxygen) or NH and
Y has either the meaning —CH₂—Ar
   wherein
   Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   or Y denotes the group gp 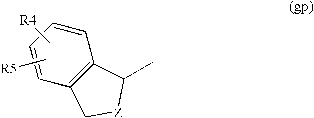
   wherein
   Z has the meaning —CHR8- or —CHR8-CHR9-
   where in Ar and/or in the group gp
   R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxy,
   R6 is hydrogen, 1-4C-alkyl or halogen and
   R7 is hydrogen, 1-4C-alkyl or halogen,
   R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
   R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino
   and wherein
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      with the proviso that R3 does not have the meaning hydrogen or halogen when Y denotes —CH₂—Ar, and the salts of these compounds.

In one aspect, the invention relates to compounds of the formula 1a 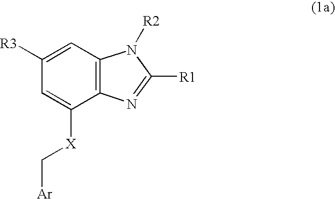
in which
R1 is hydrogen, 1-4C-alkyl, 3-7C-cycloalkyl, 3-7C-cycloalkyl-1-4C-alkyl, 1-4C-alkoxy, 1-4C-alkoxy- 1-4C-alkyl, 1-4C-alkoxycarbonyl, 2-4C-alkenyl, 2-4C-alkynyl, fluoro-1-4C-alkyl, hydroxy-1-4C-alkyl, mono- or di-1-4C-alkylamino or 1-4C-alkylcarbonyloxy-1-4C-alkyl
R2 is hydrogen, 1-4C-alkyl, aryl, 3-7C-cycloalkyl, 3-7C-cycloalkyl-1-4C-alkyl, 1-4C-alkoxycarbonyl, mono- or di-1-4C-alkylamino1-4C-alkylcarbonyl, hydroxy-1-4C-alkyl, fluoro-2-4C-alkyl, aryl-1-4C-alkoxy-1-4C-alkyl, hydroxy or 1-4C-alkoxy
R3 is hydrogen, halogen, fluoro-1-4C-alkyl, carboxyl, —CO-1-4C-alkoxy, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, cyano, the group —CO—NR31R32, the group SO₂—NR31R32 or the group Het,
   where
   R31 is hydrogen, hydroxyl, 1-7C-alkyl, hydroxy-1-4C-alkyl, 1-4C-alkoxy-1-4C-alkyl or 3-7C-cycloalkyl, amino and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group and
   Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol and tetrazol
   where
   R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R34 is hydrogen, 1-4C-alkyl, 1-4C-alkoxy, 1-4C-alkoxycarbonyl, halogen, trifluoromethyl or hydroxy,
   R35 is hydrogen, 1-4C-alkyl, 1-4C-alkoxy, 1-4C-alkoxycarbonyl, halogen, trifluoromethyl or hydroxy,
X is O (oxygen) or NH and
Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   where
   R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxy,
   R6 is hydrogen, 1-4C-alkyl or halogen and
   R7 is hydrogen, 1-4C-alkyl or halogen,
   and wherein
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      with the proviso that R3 does not have the meaning hydrogen or halogen when Y denotes —CH₂—Ar and R2 denotes hydrogen, 1-4C-alkyl or 3-7C-cycloalkyl-1-4C-alkyl,
      and the salts of these compounds

In another aspect, the invention relates to compounds of the formula 1a

in which

R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is hydrogen, 1-4C-alkyl, aryl, 3-7C-cycloalkyl, 3-7C-cycloalkyl-1-4C-alkyl, 1-4C-alkoxycarbonyl, -mono- or di-1-4C-alkylamino1-4C-alkylcarbonyl, hydroxy-1-4C-alkyl, fluoro-2-4C-alkyl, aryl-1-4C-alkoxy-1-4C-alkyl
R3 is fluoro-1-4C-alkyl, carboxyl, —CO-1-4C-alkoxy, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the group —CO—NR31R32,
   where
   R31 is hydrogen, hydroxyl, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl or 1-4C-alkoxy-1-4C-alkyl, or where
   R31 and R32 together, including the nitrogen atom to which both are bonded, are a pyrrolidino, piperidino, piperazino, N-1-4C-alkylpiperazino, morpholino, aziridino or azetidino group,
X is O (oxygen) or NH and
Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   where
   R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxy,
   R6 is hydrogen, 1-4C-alkyl or halogen and
   R7 is hydrogen, 1-4C-alkyl or halogen,
   and wherein
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      and the salts of these compounds.

In another aspect, the invention relates to compounds of the formula 1a

in which

R1 is hydrogen, 1-4C-alkyl, 3-7C-cycloalkyl, 3-7C-cycloalkyl-1-4C-alkyl, 1-4C-alkoxy, 1-4C-alkoxy- 1-4C-alkyl, 1-4C-alkoxycarbonyl, 2-4C-alkenyl, 2-4C-alkynyl, fluoro-1-4C-alkyl or hydroxy-1-4C-alkyl,
R2 is hydrogen, 1-4C-alkyl, aryl, 3-7C-cycloalkyl, 3-7C-cycloalkyl-1-C-alkyl, 1-4C-alkoxycarbonyl, mono- or di-1-4C-alkylamino1-4C-alkylcarbonyl, hydroxy-1-4C-alkyl or fluoro-2-4C-alkyl
R3 is fluoro-1-4C-alkyl, carboxyl, —CO-1-4C-alkoxy, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the group —CO—NR31R32,
   where
   R31 is hydrogen, hydroxyl, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl, or where
   R31 and R32 together, including the nitrogen atom to which both are bonded, are a pyrrolidino, piperidino, piperazino, N-1-4C-alkylpiperazino or morpholino group,
X is O (oxygen) or NH and
Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   where
   R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxy,
   R6 is hydrogen, 1-4C-alkyl or halogen and
   R7 is hydrogen, 1-4C-alkyl or halogen,
   and wherein
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      and the salts of these compounds.

In another aspect, the invention relates to compounds of the formula 1b 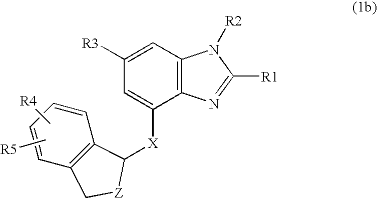
in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl, mono- or di-1-4C-alkylamino or 1-4C-alkylcarbonyloxy-1-4C-alkyl
R2 is hydrogen, 1-4C-alkyl, aryl, 3-7C-cycloalkyl, 3-7C-cycloalkyl-1-4C-alkyl, 1-4C-alkoxycarbonyl, mono- or di-1-4C-alkylamino1-4C-alkylcarbonyl, hydroxy-1-4C-alkyl, fluoro-2-4C-alkyl, aryl-1-4C-alkoxy-1-4C-alkyl, hydroxy or 1-4C-alkoxy
R3 is hydrogen, halogen, fluoro-1-4C-alkyl, carboxyl, —CO-1-4C-alkoxy, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, cyano, group —CO—NR31R32, the group SO₂—NR31R32 or the group Het,
   where
   R31 is hydrogen, hydroxyl, 1-7C-alkyl, hydroxy-1-4C-alkyl, 1-4C-alkoxy-1-4C-alkyl or 3-7C-cycloalkyl, amino and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group and
   Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol and tetrazol
   where
   R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R34 is hydrogen, 1-4C-alkyl, 1-4C-alkoxy, 1-4C-alkoxycarbonyl, halogen, trifluoromethyl or hydroxy,
   R35 is hydrogen, 1-4C-alkyl, 1-4C-alkoxy, 1-4C-alkoxycarbonyl, halogen, trifluoromethyl or hydroxy,
R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, 1-4C-alkoxy, 1-4C-alkoxycarbonyl, halogen, trifluoromethyl or hydroxy,
X is O (oxygen) or NH and
Z has the meaning —CHR8- or —CHR8-CHR9-
   where
   R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy,
   R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy,
   and wherein
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      and the salts of these compounds.

In another aspect, the invention relates to compounds of the formula 1b

in which

R1 is hydrogen, 1-4C-alkyl, 3-7C-cycloalkyl, 3-7C-cycloalkyl-1-4C-alkyl, 1-4C-alkoxy, 1-4C-alkoxy- 1-4C-alkyl, 1-4C-alkoxycarbonyl, 2-4C-alkenyl, 2-4C-alkynyl, fluoro-1-4C-alkyl or hydroxy-1-4C-alkyl,
R2 is hydrogen, 1-4C-alkyl, aryl, 3-7C-cycloalkyl, 3-7C-cycloalkyl-1-4C-alkyl, 1-4C-alkoxycarbonyl, mono- or di-1-4C-alkylamino1-4C-alkylcarbonyl, hydroxy-1-4C-alkyl, fluoro-2-4C-alkyl, aryl-1-4C alkoxy-1-4C-alkyl
R3 is hydrogen, halogen, fluoro-1-4C-alkyl, carboxyl, —CO-1-4C-alkoxy, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the group —CO—NR31R32,
   where
   R31 is hydrogen, hydroxyl, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, 1-4C-alkoxy, 1-4C-alkoxycarbonyl, halogen, trifluoromethyl or hydroxy,
X is O (oxygen) or NH and
Z has the meaning —CHR8- or —CHR8-CHR9-
   where
   R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy,
   R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy,
   and wherein
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      and the salts of these compounds.

In another aspect, the invention relates to compounds of the formula 1b

in which

R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is hydrogen, 1-4C-alkyl, aryl, 3-7C-cycloalkyl, 3-7C-cycloalkyl-1-4C-alkyl, 1-4C-alkoxycarbonyl, mono- or di-1-4C-alkylamino1-4C-alkylcarbonyl, hydroxy-1-4C-alkyl or fluoro-2-4C-alkyl,
R3 is hydrogen, halogen, fluoro-1-4C-alkyl, carboxyl, —CO-1-4C-alkoxy, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the group —CO—NR31R32,
   where
   R31 is hydrogen, hydroxyl, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino or morpholino group,
R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, 1-4C-alkoxy, 1-4C-alkoxycarbonyl, halogen, trifluoromethyl or hydroxy,
X is O (oxygen) or NH and
Z has the meaning —CHR8- or —CHR8-CHR9-
   where
   R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-1-4C-alkoxy-1-4C-alkoxy-1-4C-alkoxy, 3-7C-cycloalkoxy-1-4C-alkoxy, 3-7C-cycloalkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
   R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
   and wherein
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      and the salts of these compounds.

The compounds of the formula 1b have up to three chiral centers in the parent structure. The invention thus relates to all conceivable stereoisomers in any desired mixing ratio to one another, including the pure enantiomers, which are a preferred subject of the invention.

Among the compounds of the formula 1a, preferred compounds are those of the formula 1a-1 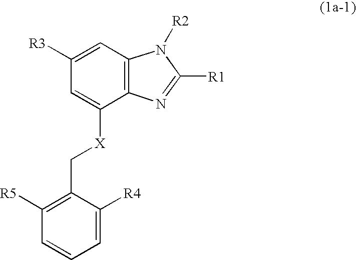
in which
R1 is hydrogen, 1-4C-alkyl, 3-7C-cycloalkyl, hydroxy-1-4C-alkyl or 1-4C-alkoxy-1-4C-alkyl,
R2 is hydrogen, 1-4C-alkyl, hydroxy, 1-4C-alkoxy or aryl-1-4C-alkoxy-1-4C-alkyl
R3 is carboxyl, —CO-1-4C-alkoxy, hydroxy-1-4C-alkyl, 1-4C-alkoxy-1-4C-alkyl, cyano, the group —CO—NR31R32, the group SO₂—NR31R32 or the group Het,
   where
   R31 is hydrogen, 1-7C-alkyl, hydroxy-1-4C-alkyl, 1-4C-alkoxy-1-4C-alkyl, 3-7C-cycloalkyl or amino and
   R32 is hydrogen or 1-7C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group and
   Het is a heterocyclic residue, substituted by R33, R34 and R35, selected from the group consisting of oxadiazol, dihydrooxazol and dihydroimidazol,
   where
   R33 is hydrogen or 1-4C-alkyl,
   R34 is hydrogen or 1-4C-alkyl
   R35 is hydrogen or 1-4C-alkyl
R4 is hydrogen, 1-4C-alkyl, hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
R5 is hydrogen, 1-4C-alkyl or 1-4C-alkoxy and
X is O (oxygen) or NH,
   and the salts of these compounds.

Among the compounds of the formula 1a, particularly preferred compounds are those of the formula 1a-1

where

R1 is hydrogen, 1-4C-alkyl or hydroxy-1-4C-alkyl,
R2 is hydrogen or 1-4C-alkyl,
R3 is carboxyl, —CO-1-4C-alkoxy, hydroxy-1-4C-alkyl, 1-4C-alkoxy-1-4C-alkyl, or the group —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen or 1-7C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
R5 is hydrogen, 1-4C-alkyl or 1-4C-alkoxy and
X is O (oxygen) or NH,
   and the salts of these compounds.

Particularly preferred compounds of the formula 1a-1 are those, in which
R1 is 1-4C-alkyl,
R2 is 1-4C-alkyl,
R3 is carboxyl, —CO-1-4C-alkoxy, 1-4C-alkoxy-1-4C-alkyl or the group —CO—NR31R32,
   where
   R31 is hydrogen, 1-4C-alkyl, hydroxy-1-4C-alkyl, 1-4C-alkoxy-1-4C-alkyl or 3-7C-cycloalkyl and
   R32 is hydrogen or 1-4C-alkyl,
R4 is 1-4C-alkyl or 1-4C-alkylcarbonylamino,
R5 is 1-4C-alkyl,
X is O (oxygen) or NH,
and their salts.

Among the compounds of the formula 1b, compounds of the formula 1 b-1 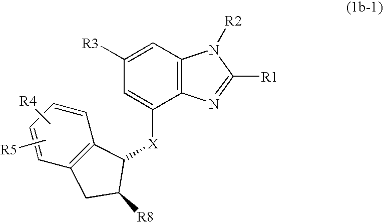
are preferred.

Preferred exemplary compounds of the formula 1 b-1 are those, in which
R1 is 1-4C-alkyl or 3-7C-cycloalkyl,
R2 is hydrogen or 1-4C-alkyl,
R3 is carboxyl, —CO-1-4C-alkoxy, 1-4C-alkoxy-1-4C-alkyl, 1-4C-alkoxy-1-4C-alkoxy-1-4C-alkyl or the group —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, 1-4C-alkoxy or halogen,
R5 is hydrogen or 1-4C-alkyl,
R8 is hydroxyl, 1-4C-alkoxy, oxo-substituted 1-4C-alkoxy, 3-7C-cycloalkoxy, 3-7C-cycloalkyl-1-4C--alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy
X is O (oxygen) or NH,
   and their salts.

Particularly preferred exemplary compounds of the formula 1b-1 are those, in which
R1 is 1-4C-alkyl,
R2 is hydrogen or 1-4C-alkyl,
R3 is carboxyl, —CO-1-4C-alkoxy, 1-4C-alkoxy-1-C-alkyl, 1-4C-alkoxy-1-4C-alkoxy-1-4C-alkyl or the group —CO—NR31R32,
   where
   R31 is hydrogen, 1-7C-alkyl, hydroxy-1-4C-alkyl, 1-4C-alkoxy-1-4C-alkyl or 3-7C-cycloalkyl
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, 1-4C-alkoxy or halogen,
R5 is hydrogen or alkyl,
R8 is hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy
X is O (oxygen) or NH,
   and their salts.

Still particularly preferred exemplary compounds of the formula 1b-1 are those, in which
R1 is 1-4C-alkyl,
R2 is hydrogen or 1-4C-alkyl,
R3 is carboxyl, —CO-1-4C-alkoxy, 1-4C-alkoxy-1-4C-alkyl, 1-4C-alkoxy-1-4C-alkoxy-1-4C-al group —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, 1-4C-alkoxy or halogen,
R5 is hydrogen,
R8 is hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy
X is O (oxygen) or NH,
   and their salts.

Preferred compounds are those of the formula 1a-1.

Particularly preferred are the compounds given as final products of formula 1 in the examples, and the salts of these compounds.

The compounds according to the invention can be synthesised from corresponding starting compounds, for example according to the reaction schemes given below. The synthesis is carried out in a manner known to the expert, for example as described in more detail in the following examples.

The starting compounds are known, for example from M. W. Lovell, S. G. Schulman, Anal. Chem. 1983, 55, 963-965 (e.g. 4-bromo-6-nitro-1,2-phenylenediamine). 6-Halo, 4-nitro-substituted benzimidazoles are known in literature, for example 6-chloro-2-methyl-4-nitro-1 (3)H-benzimidazole (Gillespie et al., J. Org. Chem. 1960, 25, 942) or they can be prepared using analogous process steps. 1,2-Epoxyindan is described for example in W. F. Whitmore; A. I. Gebhart, J. Am. Chem. Soc. 1942, 64, 912. In general, substituted alkyl-, alkoxy- or halogeno-epoxyindanes can be prepared from the corresponding substituted indenes by methods known from literature (e.g. epoxidation). The compounds of the general formula 1a can be obtained by reacting substituted benzimidazoles of formula I with compounds of formula II as depicted in scheme 1.

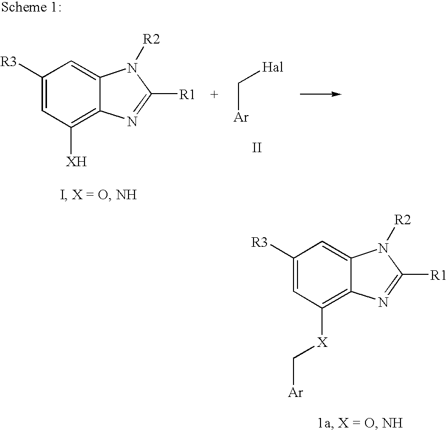

Analogously, compounds of the general formula 1b are obtained by reacting substituted benzimidazoles of formula I with epoxyindanes III, carrying any desired substituent R4 and R5 (cf. scheme 2 for a compound 1b-1).

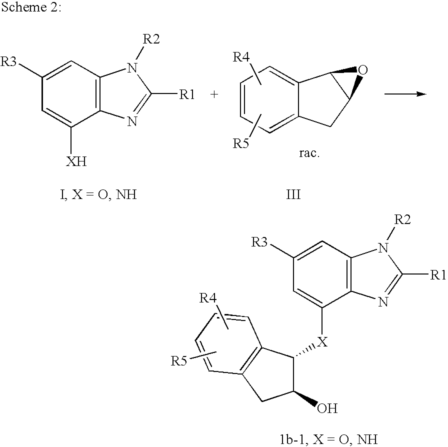

The reaction steps outlined above are carried out in a manner known per se, e.g. as described in more detail in the examples. The derivatization, if any, of the compounds obtained according to the above Scheme 1 and 2 (e.g. conversion of a group R3 into another group R3, or of R2=H into another group R2, or conversion of the hydroxyl group into an alkoxy or ester group) is likewise carried out in a manner known per se. If compounds where R3=—CO-1-4C-alkoxy or R3=—CO—NR31R32 are desired, an appropriate derivatization can be performed in a manner known per se (e.g. metal catalysed carbonylation of the corresponding bromo compound or conversion of an ester into an amide) at the stage of the benzimidazoles of formula I (scheme 1 and 2) or more conveniently at a later point in time.

The following examples serve to illustrate the invention in greater detail without restricting it. Likewise, further compounds of the formula 1 whose preparation is not described explicitly can be prepared in an analogous manner or in a manner familiar per se to the person skilled in the art using customary process techniques. The abbreviation min stands for minute(s), h for hour(s) and m.p. for melting point.

### EXAMPLES

I. Starting Materials

### A. 2-Benzyloxy-4-bromo-6-nitro-aniline

To a suspension of 50 g (325 mmol) 2-amino-3-nitrophenol, 45 g (325 mmol) potassium carbonate and 2 g (13 mmol) sodium iodide in 400 ml ethanol were added 47 ml (408 mmol) benzyl chloride and the mixture was heated to 80° C. After 2 h, the reaction mixture was cooled down and the solvent was evaporated. The residue was dissolved in ethyl acetate and extracted with water. The organic layer was dried over anhydrous magnesium sulphate and evaporated. Coevaporation with dichloromethane led to a dark brown oily residue which was dissolved in 400 ml acetonitrile. After addition of 63.4 g (356 mmol) N-bromosuccinimide, the reaction mixture was refluxed for 1 h. After cooling down, 400 g of silica gel were added and the mixture was evaporated to dryness. The resulting solid was purified by column chromatography on silica gel using ethyl acetate:light petroleum ether (4:1). Evaporation of the eluent left a solid which was recrystallized from ethyl acetate/n-heptane to give 62 g (59%) of the title compound as a red solid (m.p. 90° C.).

### B. N-Acetyl-N-(2-benzyloxy-4-bromo-6-nitro-phenyl)-acetamide

A suspension of 20 g (62 mmol) 2-benzyloxy-4-bromo-6-nitro-aniline in 120 ml acetic anhydride and 2 ml methanesulphonic acid was heated to 120° C. After complete reaction (15 min), excess acetic anhydride was evaporated. The residue was dissolved in dichloromethane/water and neutralized with 6N aqueous sodium hydroxide. The organic layer was separated, dried over anhydrous magnesium sulphate and evaporated. Crystallization of the residue from ethyl acetate/n-heptane yielded 23.2 g (92%) of the title compound as a beige solid (m.p. 148° C.).

### C. N-(2-Amino-6-benzyloxy-4-bromo-phenyl)-acetamide

A suspension of 23 g (56 mmol) N-acetyl-N-(2-benzyloxy-4-bromo-6-nitro-phenyl)-acetamide, 5.5 g (34 mmol) iron(III) chloride and 13.8 g activated charcoal in 600 ml methanol was heated to reflux. To the reaction mixture were added 28 ml hydrazine hydrate (95%) to maintain gentle reflux. After complete reaction (2 h), the mixture was cooled down and filtered through celite. The filter cake was washed thoroughly with dichloromethane/methanol and the filtrate was evaporated to dryness. The residue was partitioned between dichloromethane/methanol and water. The organic layer was washed with brine, dried over anhydrous magnesium sulphate and evaporated. The residue was recrystallized from boiling ethyl acetate/n-heptane to give 12.3 g (65%) of the title compound as a colourless solid (m.p. 185° C.).

### D. N-(2-Benzyloxy-4-bromo-6-dimethylamino-phenyl)-acetamide

A suspension of 5.0 g (15 mmol) N-(2-amino-6-benzyloxy-4-bromo-phenyl)-acetamide in 80 ml methanol and 34 ml formaldehyde (37%) was acidified with saturated methanolic hydrogen chloride to give a clear yellow solution. To the solution were added 1.5 g (24 mmol) sodium cyanoborohydride in small portions. After complete reaction (15 min), the mixture was neutralized with aqueous sodium hydrogen carbonate and extracted with dichloromethane. The organic layer was dried over anhydrous magnesium sulphate and evaporated. Crystallization of the residue from ethyl acetate/n-heptane yielded 4.3 g (79%) of the title compound as a colourless solid (m.p. 177° C.).

### E. 4-Benzyloxy-6-bromo-1,2-dimethyl-1H-benzimidazole

A suspension of 26.2 g (72 mmol) N-(2-benzyloxy-4-bromo-6-dimethylaminophenyl)-acetamide in 180 ml phosphoryl chloride was heated to 70° C. for 24 h. After the reaction was complete, excess phosphoryl chloride was evaporated. The residue was suspended in dichloromethane and carefully neutralized with 6N aqueous potassium hydroxide and water. The organic layer was separated, dried over anhydrous magnesium sulphate and evaporated. Crystallization of the residue from ethyl acetate yielded 15.1 g (63%) of the title compound as a colourless solid (m.p. 177-179° C.).

### F. Ethyl 4-hydroxy-1,2-dimethyl-1H-benzimidazole-6-carboxylate

A suspension of 12.0 g (37 mmol) ethyl 4-benzyloxy-1,2-dimethyl-1H-benzimidazole-6-carboxylate in 100 ml ethanol was hydrogenated over 1 g 10% Pd/C (50° C., 5 bar H₂) for 16 h. The catalyst was filtered off and the filtrate was evaporated. The residue was crystallized from ethanol/diethyl ether to give 5.91 g (69%) of the title compound as a colourless solid (m.p. 272-273° C.).

### G. Methyl 4-hydroxy-1,2-dimethyl-1H-benzimidazole-6-carboxylate

A suspension of 8.5 g (27 mmol) methyl 4-benzyloxy-1,2-dimethyl-1H-benzimidazole-6-carboxylate in 100 ml methanol was hydrogenated over 0.7 g 10% Pd/C (30° C., 1 bar H₂) for 2 h. The catalyst was filtered off, washed several times with hot methanol and the filtrate was evaporated. The residue was crystallized from methanol/diethyl ether to give 6 g (99%) of the title compound as a colourless solid (m.p. 286° C.).

### H. 4-Hydroxy-6-methoxymethyl-1,2-dimethyl-1H-benzimidazole

A solution of 1.2 g (4.1 mmol) 4-benzyloxy-6-methoxymethyl-1,2-dimethyl-1H-benzimidazole in 12 ml methanol was hydrogenated over 0.12 g 10% Pd/C (1 bar H₂) for 16 h. The catalyst was filtered off and the filtrate was evaporated. The residue was crystallized from ethyl acetate/light petroleum ether to give 0.83 g (99%) of the title compound as a colourless solid (m.p. 219-220° C.).

### I. 6-(N,N-Dimethylaminocarbonyl)-4-hydroxy-1,2-dimethyl-1H-benzimidazole

A solution of 2.3 g (7.1 mmol) 4-benzyloxy-6-(N,N-dimethylaminocarbonyl)-1,2-dimethyl-1H-benzimidazole in 80 ml methanol was hydrogenated over 0.3 g 10% Pd/C (1 bar H₂) for 16 h. The catalyst was filtered off and the filtrate was evaporated. The residue was crystallized from acetone to give 1.2 g (71%) of the title compound as a colourless solid (m.p. 248° C.).

### J. 6-Bromo-2-methyl-4-nitro-1(3)H-benzimidazole

To a suspension of 65 g (0.28 mol) 5-bromo-3-nitro-1,2-phenylenediamine in 600 ml ethanol were added 140 ml 5N hydrochloric acid. The reaction mixture was hetaed to reflux and 58 ml (0.56 mol) 2,4-pentanedione were added in one portion. After 1 h, the mixture was cooled down, poured into 500 ml water and neutralized with conc. ammonia. The precipitate was collected, washed thoroughly with water and dried over phosphorus pentoxide to give 70.8 g (99%) of the title compound (m.p. 229-231° C.).

### K. 6-Bromo-4-nitro-1(3)H-benzimidazole

To a suspension of 14.25 g (61.4 mmol) 5-bromo-3-nitro-1,2-phenylenediamine in 120 ml 4N hydrochloric acid were added 4.65 ml (123 mmol) formic acid. The reaction mixture was heated to 120° C. After 1.5 h, the mixture was cooled down and neutralized with conc. ammonia. The precipitate was collected, washed with water and dried over phosphorus pentoxide. Recrystallization from ethanol and activated charcoal yielded 9.79 g (66%) of the title compound (m.p. 249° C.).

### L. 6-Bromo-2-methoxymethyl nitro-1(3)H-benzimidazole

A suspension of 1 g (4.3 mmol) 5-bromo-3-nitro-1,2-phenylenediamine in 4 ml methoxyacetic acid was heated to 110° C. for 16 h. The mixture was poured into icewater, neutralized with 6N aqueous sodium hydroxide and extracted with dichloromethane. The organic layer was separated, dried over anhydrous magnesium sulphate and evaporated. Purification of the residue by crystallization from ethyl acetate and activated charcoal yielded 0.9 g (73%) of the title compound as a colourless solid (m.p. 173° C.).

### M. 6-Bromo-1-methyl-4-nitro-1H-benzimidazole

A suspension of 5.12 g (21.2 mmol) 6-bromo-4-nitro-1 (3)H-benzimidazole and 4.6 g (33.3 mmol) potassium carbonate in 200 ml acetone was stirred 30 min and 1.54 ml (24.7 mmol) methyl iodide were then added. After stirring 18 h at ambient temperature, excess solvent was evaporated and the residue was partitioned between dichloromethane and water. The organic layer was separated, dried over anhydrous magnesium sulphate and evaporated. Purification of the residue by crystallization from ethyl acetate yielded 2.7 g (50%) of the title compound as a colourless solid (m.p. 198° C.).

### N. 6-Bromo-2-methoxymethyl-1-methyl-4-nitro-1H-benzimidazole

To a suspension of 13 g (45.4 mmol) 6-bromo-2-methoxymethyl-4-nitro-1 (3)H-benzimidazole and 12.6 g (90.9 mmol) potassium carbonate in 130 ml acetone were added 6.8 g (47.7 mmol) methyl iodide and the mixture was stirred for 18 h at ambient temperature. The thick suspension was poured into 200 ml water and extracted with dichloromethane. The organic layer was dried over anhydrous magnesium sulphate and evaporated. Purification of the residue by crystallization from diisopropyl ether yielded 13.6 g (99%) of the title compound as a yellow solid (m.p. 145-147° C.).

### O. 1-Benzyloxymethyl-6-bromo-2-methyl-4-nitro-1H-benzimidazole

To a suspension of 0.26 g (6.4 mmol) sodium hydride (60% dispersion in mineral oil) in 10 ml N,N-dimethylformamide was slowly added a solution of 1.5 g (5.9 mmol) 6-bromo-2-methyl-4-nitro-1 (3)H-benzimidazole in 5 ml N,N-dimethylformamide at 0° C. After 1 h at 0° C., 2.29 g (8.8 mmol) benzyloxy-methylchloride (60%) were added over 20 min. When the reaction was finished (1 h), 10 ml water were carefully added and the mixture was partitioned between dichloromethane and water. The organic layer was dried over anhydrous magnesium sulphate and evaporated. Purification of the residue by column chromatography on silica gel using ethyl acetate:triethylamine (95:5) gave an oil which was crystallized from diisopropyl ether to yield 1.16 g (53%) of the title compound as a yellow solid (m.p. 106-109° C.).

### P. 6-Bromo-1,2-dimethyl-4-nitro-1H-benzimidazole

To a suspension of 4.3 g (107 mmol) sodium hydride (60% dispersion in mineral oil) in 25 ml N,N-dimethylformamide was slowly added a solution of 25 g (98 mmol) 6-bromo-2-methyl-4-nitro-1 (3)H-benzimidazole in 100 ml N,N-dimethylformamide at 0° C. After 30 min at 0° C., 15.2 g (107 mmol) methyl iodide were added over 20 min. When the reaction was finished (45 min), 200 ml water were carefully added and the mixture was stirred 1 h at ambient temperature. The precipitate was collected, washed thoroughly with water and dried over phosphorus pentoxide. Recrystallization from methanol yielded 19.6 g (74%) of the title compound as a colourless solid (m.p. 193-195° C.).

### Q. 4-Amino-6-bromo-1-methyl-1H-benzimidazole

To a suspension of 10 g (39 mmol) 6-bromo-1-methyl-4-nitro-1H-benzimidazole in 100 ml methanol were added 7.6 g (46.9 mmol) iron(III) chloride and 2.5 g activated charcoal. The reaction mixture was heated to 80° C. and 9.5 ml hydrazine hydrate (95%) were slowly added. After refluxing 2 h, a further amount of 2 ml hydrazine hydrate (95%) was added. After 5 h, the hot reaction mixture was filtered through celite and the filter cake was washed with methanol and dichloromethane. The filtrate was evaporated to leave a solid, which was purified by column chromatography on silica gel using toluene:dioxane:methanol (6:3.6:0.4). Crystallization from diisopropyl ether yielded 6.8 g (77%) of the title compound as a light yellow solid (m.p. 170° C.).

### R. 4-Amino-6-bromo-2-methoxymethyl-1-methyl-1H-benzimidazole

To a suspension of 8.5 g (28.3 mmol) 6-bromo-2-methoxymethyl-1-methyl-4-nitro-1H-benzimidazole in 150 ml methanol were added 5.51 g (34 mmol) iron(III) chloride, 2.5 g activated charcoal and 6.9 ml hydrazine hydrate (95%). After refluxing 2 h, a further amount of 4 ml hydrazine hydrate (95%) was added. After 4 h, the hot reaction mixture was filtered through celite and the filter cake was washed with methanol and dichloromethane. The filtrate was evaporated and the residue was purified by column chromatography on silica gel using dichloromethane:methanol (20:1). Crystallization from diisopropyl ether yielded 5.5 g (72%) of the title compound as a colourless solid (m.p. 138-140° C.).

### S. 4-Amino-1-benzyloxymethyl-4-bromo-2-methyl-1H-benzimidazole

To a suspension of 10 g (26.6 mmol) 1-benzyloxymethyl-6-bromo-2-methyl-4-nitro-1H-benzimidazole and 1 g activated charcoal in 150 ml methanol were added 5.2 g (31.9 mmol) iron(III) chloride and 6.5 ml hydrazine hydrate (95%). After refluxing 1 h, a further amount of 3 ml hydrazine hydrate (95%) was added. After 6 h, the hot reaction mixture was filtered through celite and the filter cake was washed with hot methanol. The filtrate was evaporated to leave a yellow solid which was purified by column chromatography on silica gel using dichloromethane:methanol (9:1). Crystallization from diisopropyl ether yielded 6.68 g (73%) of the title compound as a light yellow solid (m.p. 142° C.).

### T. 4-Amino-6-bromo-1,2-dimethyl-1H-benzimidazole

To a solution of 19 g (70 mmol) 6-bromo-1,2-dimethyl-4-nitro-1H-benzimidazole in 250 ml methanol were added 13.7 g (84 mmol) iron(III) chloride and 6 g activated charcoal. The reaction mixture was heated to 80° C. and 17 ml hydrazine hydrate (95%) were slowly added. After refluxing 3 h, the hot reaction mixture was filtered through celite and the filter cake was washed with methanol and dichloromethane. The filtrate was evaporated to leave a suspension, which was treated with n-heptane. The precipitate was collected, washed with n-heptane and dried to give 13.3 g (79%) of the title compound as a solid (m.p. 206-209° C.).

### U. 6-Bromo-4-(2-ethyl-6-methyl-benzylamino)-1,2-dimethyl-1H-benzimidazole

To a suspension of 12 g (50 mmol) 4-amino-6-bromo-1,2-dimethyl-1H-benzimidazole and 8.8 g (52 mmol) 2-ethyl-6-methyl-benzyl chloride in 220 ml acetone were added 5.5 g (52 mmol) sodium carbonate and 1.5 g (10 mmol) sodium iodide. After 3 h stirring at 45° C., the reaction mixture was poured into 400 ml water and extracted with ethyl acetate. The organic layer was separated, dried over anhydrous magnesium sulphate and evaporated. Purification of the residue by column chromatography on silica gel using dichloromethane:methanol (100:3) and crystallization with n-heptane yielded 15.6 g (84%) of the title compound as a colourless solid (m.p. 145-147° C.).

### V. 6-Bromo-4-(2,6-dimethyl-benzylamino)-1,2-dimethyl-benzimidazole

To a suspension of 0.97 g (4 mmol) 4-amino-6-bromo-1,2-dimethyl-1H-benzimidazole and 0.66 g (4.2 mmol) 2,6-dimethyl-benzyl chloride in 25 ml acetonitrile were added 0.56 g (4 mmol) potassium carbonate and 70 mg (0.4 mmol) potassium iodide. After 1 h stirring at 65° C., 2 ml 1N aqueous ammonia were added and the reaction mixture was evaporated. The residue was partitioned between dichloromethane and water. The organic layer was separated, dried over anhydrous magnesium sulphate and evaporated. Purification of the residue by column chromatography on silica gel using ethyl acetate:light petroleum ether (1:1) and crystallization from ethyl acetate/n-heptane yielded 0.93 g (64%) of the title compound as a colourless solid (m.p. 185° C.).

### W. 1-Benzyloxymethyl-6-bromo-4-(2-ethyl-6-methyl-benzylamino)-2-methyl-1H-benzimidazole

To a suspension of 6.5 g (18.8 mmol) 4-amino-1-benzyloxymethyl-6-bromo-2-methyl-1H-benzimidazole and 3.32 g (19.7 mmol) 2-ethyl-6-methyl-benzyl chloride in 120 ml acetonitrile were added 3.0 g (28.2 mmol) sodium carbonate and 0.56 g (3.75 mmol) sodium iodide. After 3 h reflux, the reaction mixture was poured into 300 ml water and extracted with dichloromethane. The organic layer was separated, dried over anhydrous magnesium sulphate and evaporated. Purification of the residue by column chromatography on silica gel using ethyl acetate:light petroleum ether (2:3) and crystallization from diisopropyl ether yielded 6.88 g (77%) of the title compound as a colourless solid (m.p. 117-119° C.).

### X. 6-Bromo-4-(2-ethyl-6-methyl-benzylamino)-1-methyl-1H-benzimidazole

To a suspension of 2.0 g (8.9 mmol) 4-amino-6-bromo-1-methyl-1H-benzimidazole and 1.6 g (9.5 mmol) 2-ethyl-6-methyl-benzyl chloride in 40 ml acetonitrile were added 1.4 g (13.1 mmol) sodium carbonate and 0.3 g (2 mmol) sodium iodide. After 1 h at 70° C., the mixture was partitioned between dichloromethane and water. The organic layer was separated, dried over anhydrous magnesium sulphate and evaporated. Purification of the residue by column chromatography on silica gel using dichloromethane:methanol (100:1) yielded 2.0 g (63%) of the title compound as a colourless solid (m.p. 132-134° C.).

### Y. 6-Bromo-4-(2-ethyl-6-methyl-benzylamino)-2-methoxymethyl-1-methyl-1H-benzimidazole

To a suspension of 6.8 g (25.2 mmol) 4-amino-6-bromo-2-methoxymethyl-1-methyl-1H-benzimidazole and 4.46 g (26.4 mmol) 2-ethyl-6-methyl-benzylchloride in 70 ml acetonitrile were added 7.0 g (50.3 mmol) potassium carbonate and a catalytic amount of potassium iodide. After 5 h at 70° C., the reaction mixture was poured into 200 ml water and extracted with dichloromethane. The organic layer was separated, dried over anhydrous magnesium sulphate and evaporated. Purification of the residue by column chromatography on silica gel using ethyl acetate:light petroleum ether (1:1) and crystallization from diisopropyl ether yielded 5.46 g (54%) of the title compound as a colourless solid (m.p. 110-112° C.).

### Z. 6-Bromo-4-(2,6-dimethyl-benzylamino)-2-methoxymethyl-1-methyl-1H-benzimidazole

To a suspension of 2.5 g (9.25 mmol) 4-amino-6-bromo-2-methoxymethyl-1-methyl-1H-benzimidazole and 1.52 g (9.72 mmol) 2,6-dimethyl-benzyl chloride in 50 ml acetonitrile were added 2.56 g (18.5 mmol) potassium carbonate and a catalytic amount of potassium iodide. After 3 h at 70° C., the reaction mixture was poured into 150 ml water and extracted with dichloromethane. The organic layer was separated, dried over anhydrous magnesium sulphate and evaporated. Purification of the residue by column chromatography on silica gel using dichloromethane:methanol (100:1) and crystallization from diisopropyl ether yielded 0.84 g (23%) of the title compound as a colourless solid (m.p. 143-145° C.).

### AA. 4-Amino-6-(N,N-dimethylaminocarbonyl)-1,2-dimethyl-1H-benzimidazole

To a suspension of 3 g (12.5 mmol) 4-amino-6-bromo-1,2-dimethyl-1H-benzimidazole in 100 ml dimethylamine (2M in tetrahydrofuran) were added 280 mg (1.25 mmol) palladium(II) acetate and 2 g (7.5 mmol) triphenylphosphine. The mixture was transferred to an autoclave and carbonylated (6 bar carbon monoxide pressure, 120° C.) for 16 h. The reaction mixture was cooled down, poured into 200 ml water and 100 ml saturated aqueous ammonium chloride and extracted with dichloromethane. The organic layer was separated, washed with water, dried over anhydrous magnesium sulphate and evaporated. Purification of the residue by column chromatography on silica gel using ethyl acetate/methanol (4:1) and crystallization from diisopropyl ether yielded 0.7 g (24%) of the title compound as a colourless solid (m.p. 230-234° C.).

### BB. Cyclopropanecarboxylic acid (2-benzyloxy-4-bromo-6-nitro-phenyl)-amide

A suspension of 20.0 g (61.9 mmol) 2-benzyloxy-4-bromo-6-nitro-aniline and 11.2 ml (123.4 mmol) cyclopropanecarbonyl chloride in 130 ml dioxane was heated to 100° C. After 5 h, the flask was immersed in an ice bath, the precipitate was collected, washed with toluene and dried in vacuo to give 21.2 g (87%) of the title compound as a yellow solid (m.p. 191° C.).

### CC. Cyclopropanecarboxylic acid (2-amino-6-benzyloxy-4-bromo-phenyl)-amide

A suspension of 11.0 g (28.1 mmol) cyclopropanecarboxylic acid (2-benzyloxy-4-bromo-6-nitro-phenyl)amide, 3.0 g (18.5 mmol) iron(III) chloride and 7.4 g activated charcoal in 140 ml methanol was heated to reflux. To the reaction mixture were added 14.7 ml hydrazine hydrate (95%) to maintain gentle reflux. After complete reaction (1 h), the mixture was cooled down and filtered through celite. The filter cake was washed thoroughly with hot methanol/acetone and the filtrate was evaporated to dryness. The residue was crystallized from ethyl acetate to give 7.5 g (73%) of the title compound as a colourless solid (m.p. 207-208° C.).

### DD. 4-Benzyloxy-6-bromo-2-cyclopropyl-1-methyl-1H-benzimidazole

A suspension of 7.5 g (20.8 mmol) cyclopropanecarboxylic acid (2-amino-6-benzyloxy-4-bromo-phenyl)amide in 80 ml methanol and 13.3 ml formaldehyde (37%) was acidified with saturated methanolic hydrogen chloride to give a clear yellow solution. To the solution were added 0.75 g (11.9 mmol) sodium cyanoborohydride in small portions. After complete reaction (4 h), the mixture was poured into water and neutralized with 40% aqueous sodium hydroxide. The precipitate was collected, washed with water and dried over phosphorus pentoxide to yield 5.3 g of a colourless solid. The solid was suspended in 10 ml phosphoryl chloride and the mixture was heated to 90° C. for 2 h. After the reaction was complete, the mixture was diluted with dichloromethane and neutralized with with 6N aqueous potassium hydroxide and water. The organic layer was separated, dried over anhydrous magnesium sulphate and evaporated. Purification of the residue by column chromatography on silica gel using toluene:dioxane (10:1) and crystallization from diethyl ether yielded 2.97 g (40%) of the title compound as a colourless solid (m.p. 153-154° C.).

### EE. Ethyl 2-cyclopropyl-4-hydroxy-1-methyl-1H-benzimidazole-6-carboxylate

A suspension of 2.3 g (6.6 mmol) ethyl 4-benzyloxy-2-cyclopropyl-1-methyl-1H-benzimidazole-6-carboxylate in 23 ml methanol was hydrogenated over 0.27 g 10% Pd/C (25° C., 1 bar H₂) for 5 h. The catalyst was filtered off and the filtrate was evaporated. The residue was crystallized from ethyl acetate to give 1.5 g (88%) of the title compound as a colourless solid (m.p. 201-202° C.).

### FF. 4-Amino-N,N-dimethyl-3,5-dinitro-benzenesulfonamide

A suspension of 5.0 g (27.3 mmol) 2,6-dinitroaniline in 40 ml chlorosulphonic acid was heated to 100° C. After 2.5 h, the reaction mixture was cooled down and cautiously poured onto 1000 ml crushed ice. The suspension was extracted with dichloromethane. The organic layer was dried over anhydrous magnesium sulphate and evaporated to dryness. The residue was dissolved in 100 ml tetrahydrofuran and a solution of 25 ml dimethylamine in 25 ml tetrahydrofuran was slowly added. After 20 min, the suspension was evaporated and the residue was partitioned between dichloromethane and water. The organic layer was separated, dried over anhydrous magnesium sulphate and evaporated. Purification of the residue by crystallization from ethanol yielded 6.7 g (85%) of the title compound as an orange solid (m.p. 169-170° C.).

### GG. 3,4-Diamino-N,N-dimethyl-5-nitro-benzenesulfonamide

50 ml 2N aqueous ammonia were saturated with hydrogen sulfide at 0° C. The solution was diluted with 50 ml ethanol and 5.58 g (19.2 mmol) 4-amino-N,N-dimethyl-3,5-dinitro-benzenesulfonamide were added. After 40 min at 60° C., the mixture was diluted with water and filtered through celite. The filter cake was extracted several times with boiling dichloromethane and methanol. The combined extracts were evaporated and the residue recrystallized from ethanol/diethyl ether to yield 1.18 g (24%) of the title compound as a red solid (m.p. 211-213° C.).

### HH. 2-Methyl-7-nitro-3H-benzimidazole-5-sulfonic acid dimethylamide

A suspension of 1.0 g (3.84 mmol) 3,4-diamino-N,N-dimethyl-5-nitro-benzenesulfonamide in 20 ml ethanol and 5 ml 5N hydrochloric acid was heated to 80° C. To the reaction mixture were added 1.6 ml (15.4 mmol) 2,4-pentanedione in two portions over a period of 1 h. The solution was cooled down and neutralized with 6N aqueous sodium hydroxide. The precipitate was collected, washed with water and dried in vacuo over phosphorus pentoxide to give 0.99 g (90%) of a beige solid (m.p. 254-255° C.).

### II. 2,3-Dimethyl-7-nitro-3H-benzimidazole-5-sulfonic acid dimethylamide

A suspension of 1.87 g (6.6 mmol) 2-methyl-7-nitro-3H-benzimidazole-5-sulfonic acid dimethylamide, 1.82 g (13.2 mmol) potassium carbonate and 0.71 ml (11.5 mmol) methyl iodide in 50 ml acetone was stirred 3 h at ambient temperature. The mixture was partitioned between dichloromethane and saturated aqueous ammonium chloride. The organic layer was separated, dried over anhydrous magnesium sulphate and evaporated to dryness. Recrystallization of the residue from boiling ethyl acetate yielded 1.5 g (76%) of the title compound as a beige solid (m.p. 194-196° C.).

### JJ. 7-Amino-2,3-dimethyl-3H-benzimidazole-5-sulfonic acid dimethylamide

A suspension of 1.2 g (4.0 mmol) 2,3-dimethyl-7-nitro-3H-benzimidazole-5-sulfonic acid dimethylamide in 15 ml methanol and 5 ml acetic acid was heated to 60° C. and 1.1 g (20 mmol) iron filings were added. After 1.5 h, the solids were filtered off and extracted several times with boiling dichloromethane. The combined extracts were evaporated and the residue was crystallized from ethyl acetate/n-heptane to yield 0.99 g (92%) of the title compound as a beige solid (m.p. 255-258° C.).

### KK. N-(4-Bromo-2,6-dinitro-phenyl)acetamide

A suspension of 5.0 g (19.1 mmol) 4-bromo-2,6-dinitroaniline in 50 ml acetic anhydride and 1 ml methanesulphonic acid was stirred 3 h at 30° C. The precipitate was collected, washed with diethyl ether and dried to yield 4.8 g (83%) of the title compound as a colourless solid (m.p. 238-239° C.).

### LL. 4-Amino-6-bromo-1-hydroxy-2-methyl-1H-benzimidazole

To a suspension of 2.0 g (6.6 mmol) N-4-bromo-2,6-dinitro-phenyl)-acetamide and 0.4 g ruthenium on charcoal (5%) in 80 ml ethanol were added 1.2 ml (24.7 mmol) hydrazine hydrate (95%) over a period of 1.5 h at 60° C. The catalyst was filtered off and the filtrate was evaporated to dryness. The residue was extracted with boiling ethyl acetate to leave 1.23 g (77%) of the title compound as a light grey solid (m.p. 257-258° C.).

### MM. N-[5-Bromo-2-(2-chloro-acetylamino)-3-nitro-phenyl]-2-chloro-acetamide

To a solution of 46.4 g (200 mmol) 4-bromo-2,6-dinitroaniline in 500 ml N,N-dimethylformamide and 20 ml pyridine were slowly added 80 ml (1000 mmol) chloroacetyl chloride. After 3 h, the reaction mixture was poured into 600 ml ice water and the resulting suspension was neutralized with 6N aqueous sodium hydroxide. The precipitate was collected, washed with water and dried in vacuo to yield 74.1 g (96%) of the title compound as a beige solid (m.p. 172-173° C.).

### NN. 6-Bromo-2-chloromethyl-4-nitro-1H-benzimidazole

A suspension of 1.0 g (2.59 mmol) N-[5-bromo-2-(2-chloro-acetylamino)-3-nitro-phenyl]-2-chloro-acetamide in 25 ml 4N hydrochloric acid and 20 ml ethanol was heated to reflux. After 4 h, the reaction mixture was poured into 75 ml water and neutralized with saturated aqueous sodium hydrogen carbonate. The precipitate was collected, washed with water and dried over phosphorus pentoxide to give 0.65 g (86%) of the title compound as an orange solid (m.p. 148-150° C.).

### OO. 6-Bromo-2-chloromethyl-1-ethyl-4-nitro-1H-benzimidazole

To a solution of 39.3 g (135.3 mmol) 6-bromo-2-chloromethyl-4-nitro-1H-benzimidazole in 500 ml acetone were added 26.0 g (206.1 mmol) dimethyl sulphate and 50.0 g (362 mmol) potassium carbonate. After 1.25 h stirring at room temperature, the reaction mixture was poured into 1000 ml water. The precipitate was collected, washed with water and recrystallized from methanol to give 32.5 g (63%) of the title compound as a beige solid (m.p. 154-155° C.).

### PP. 2-Acetoxymethyl-6-bromo-1-methyl-4-nitro-1H-benzimidazole

To a solution of 30.0 g (98.5 mmol) 6-bromo-2-chloromethyl-1-methyl-nitro-1H-benzimidazole in 200 ml acetone were added 16.4 g (98.5 mmol) potassium iodide. The resulting suspension was stirred 1 h at ambient temperature and 14.5 g (147.8 mmol) potassium acetate were added. After 2 h at 40° C., the reaction mixture was poured into 500 ml water and extracted with dichloromethane. The organic layer was separated, dried over anhydrous magnesium sulphate and evaporated to dryness. Recrystallization of the residue from boiling ethanol yielded 23.9 g (74%) of the title compound as an orange solid (m.p. 143-145° C.).

### QQ. 2-Acetoxymethyl amino-6-bromo-1-methyl-1H-benzimidazole

To a suspension of 10.0 g (30.5 mmol) 2-acetoxymethyl-bromo-1-methyl-4-nitro-1H-benzimidazole in 40 ml acetic acid were added 6.8 g (122 mmol) iron filings and the reaction mixture was heated to 60° C. After 4 h, a further amount of 1.0 g (18 mmol) iron filings were added and heating was continued for 1.25 h. The precipitate was collected, dissolved in dichloromethane and extracted with water. The organic layer was separated, dried over anhydrous magnesium sulphate and evaporated to dryness. Purification of the residue by column chromatography on silica gel using ethyl acetate:light petroleum ether (7:3) and crystallization from ethyl acetate/n-heptane yielded 4.6 g (51%) of the title compound as a beige solid (m.p. 104-106° C.).

### RR. 4-Amino-6-bromo-2-hydroxymethyl-1-methyl-1H-benzimidazole

A suspension of 7.5 g (22.9 mmol) 2-acetoxymethyl-6-bromo-1-methyl-4-nitro-1H-benzimidazole in 180 ml methanol was hydrogenated over Raney nickel (25° C., 1 bar H₂) for 2.5 h. The catalyst was filtered off and the filtrate was partitioned between water and dichloromethane. The organic layer was separated, dried over anhydrous magnesium sulphate and evaporated. The residue was crystallized from ethanol/diisopropyl ether to give 1.32 g (22%) of the title compound as a solid (m.p. 216-218° C.).

### SS. 2,6-Dibromo-1-methyl-4-nitro-1H-benzimidazole

To a solution of 1.0 g (3.9 mmol) 6-bromo-1-methyl-4-nitro-1H-benzimidazole in 25 ml dichloroethane were added 5 g silica gel and 0.94 g (5.3 mmol) N-bromosuccinimide. After 2.5 h at 85° C., the mixture was cooled down and filtered. The filtrate was extracted with saturated aqueous sodium hydrogen carbonate, dried over anhydrous magnesium sulphate and evaporated. Purification of the residue by column chromatography on silica gel using ethyl acetate:light petroleum ether (1:1) and crystallization from ethyl acetate/n-heptane yielded 0.745 g (57%) of the title compound as a yellow solid (m.p. 235-236° C.).

### TT. 6-Bromo-2-(N,N-dimethylamino)-1-methyl-4-nitro-1H-benzimidazole

To a solution of 3.0 g (11.7 mmol) 6-bromo-1-methyl-4-nitro-1H-benzimidazole in 75 ml dichloroethane were added 25 g silica gel and 2.5 g (14.1 mmol) N-bromosuccinimide. After 3 h at 85° C., the mixture was cooled down and filtered. The filtrate was evaporated and the residue was dissolved in 100 ml tetrahydrofuran. A solution of 10 ml dimethylamine in 20 ml tetrahydrofuran was slowly added and the mixture was stirred for 4 h. The mixture was evaporated to ¼ of its volume and partitioned between saturated aqueous sodium hydrogen carbonate and dichloromethane. The organic layer was separated, dried over anhydrous magnesium sulphate and evaporated. Purification of the residue by crystallization from ethyl acetate yielded 1.6 g (46%) of the title compound as an orange coloured solid (m.p. 206-207° C.).

### UU. 4-Amino-6-bromo-2-(N,N-dimethylamino)-1-methyl-1H-benzimidazole

To a suspension of 1.0 g (3.3 mmol) 6-bromo-2-(N,N-dimethylamino)-1-methyl-4-nitro-1H-benzimidazole in 25 ml methanol and 8 ml acetic acid were added 0.85 g (15.2 mmol) iron powder at 65 ° C. After 2 h, the solids were filtered off and washed with dichloromethane. The filtrate was extracted with water, dried over anhydrous magnesium sulphate and evaporated. Purification of the residue by column chromatography on silica gel using ethyl acetate and crystallization from diethyl ether yielded 0.2 g (22%) of the title compound as a colourless solid (m.p. 137° C.).

### VV. 6-Bromo-1,2-dimethyl-4-(2-methyl-benzylamino)-1H-benzimidazole

To a solution of 0.9 ml (6.4 mmol) 2-methyl-benzyl chloride in 20 ml acetone were added 1.1 g (6.6 mmol) sodium iodide and the mixture was stirred 1.5 h at room temperature. The solid was filtered off and 1.5 g (4.4 mmol) 4-amino-6-bromo-1,2-dimethyl-1H-benzimidazole and 1.3 g (9.4 mmol) potassium carbonate were added to the filtrate. After 3 h at reflux, the mixture was partitioned between water and dichloromethane. The organic layer was separated, dried over anhydrous magnesium sulphate and evaporated. Purification of the residue by column chromatography on silica gel using dichloromethane:methanol (30:1) and crystallization from ethyl acetate/n-heptane yielded 1.28 g (61%) of the title compound as a colourless solid (m.p. 128-129° C.).

### WW. 6-Bromo-4-(2,4-dimethyl-furan-3-yl-methylamino)-1,2-dimethyl-1H-benzimidazole

To a suspension of 1.95 g (4.6 mmol) Dess-Martin-periodinane in 10 ml dichloromethane and 0.5 ml pyridine was dropwise added a solution of 0.5 g (4.0 mmol) (2,4-dimethyl-furan-3-yl)-methanol in 5 ml dichloromethane at room temperature. After 30 min, water and 1 ml saturated aqueous sodium sulfite were added and the mixture was extracted with dichloromethane. The organic layer was separated, dried over anhydrous magnesium sulphate and evaporated. The residue was filtered through silica gel using ethyl acetate:light petroleum ether (9:1) to leave a colourless oil after evaporation. The crude product was dissolved in 10 ml methanol and 0.5 ml acetic acid. After addition of 0.48 g (2 mmol) 4-amino-6-bromo-1,2-dimethyl-1H-benzimidazole, 0.3 g (4.8 mmol) sodium cyanoborohydride were added in three portions over a period of 2.5 h. After 2 h, saturated aqueous sodium hydrogen carbonate was added and the mixture was extracted with dichloromethane. The organic layer was dried over anhydrous magnesium sulphate and evaporated. Purification of the residue by column chromatography on silica gel using ethyl acetate and crystallization from ethyl acetate/n-heptane yielded 0.45 g (65%) of the title compound as a colourless solid (m.p. 207-208° C.).

### XX. 6-Bromo-4-(2-hydroxymethyl-6-methyl-benzylamino)-1,2-dimethyl-1H-benzimidazole

To a solution of 0.8 g (5.4 mmol) 7-methyl-3H-isobenzofuran-1-one in 15 ml dried toluene were slowly added 4.3 ml (6.5 mmol) diisobutylaluminium hydride (1.5M in toluene) at −78° C. After 1 h, the reaction mixture was quenched with 1 ml methanol and allowed to warm to room temperature. The mixture was partitioned between saturated aqueous potassium sodium tartrate and dichloromethane. The organic layer was separated, dried over anhydrous magnesium sulphate and evaporated. The oil thus obtained was dissolved in 20 ml methanol and 1 ml acetic acid. After the addition of excess sodium cyanoborohydride, the mixture was stirred 4 h at 45° C. The mixture was partitioned between water and dichloromethane and neutralized with 6N aqueous sodium hydroxide. The organic layer was separated, dried over anhydrous magnesium sulphate and evaporated. Purification of the residue by column chromatography on silica gel using ethyl acetate and crystallization from ethyl acetate/n-heptane yielded 0.35 g (24%) of the title compound as a colourless solid (m.p. 215-216° C.).

### YY. 6-Bromo-4-(2-ethyl-6-methyl-benzylamino)-2-hydroxymethyl-1-methyl-1H-benzimidazole

A suspension of 1.25 g (4.88 mmol) 4-amino-6-bromo-2-hydroxymethyl-1-methyl-1H-benzimidazole, 0.86 g (5.12 mmol) 2-ethyl-6-methyl-benzyl chloride, 1.35 g (9.76 mmol) potassium carbonate and a catalytic amount of potassium iodide in 15 ml acetonitrile was heated to 70° C. After 6 h, the reaction mixture was poured into water and extracted with dichloromethane. The organic layer was separated, dried over anhydrous magnesium sulphate and evaporated. Purification of the residue by column chromatography on silica gel using dichloromethane:methanol (100:3) and crystallization from diisopropyl ether yielded 0.54 g (29%) of the title compound as a colourless solid (m.p. 206-208° C.).

### ZZ. 6-Bromo-2-(N,N-dimethylamino)-4-(2,6-dimethyl-benzylamino)-1-methyl-1H-benzimidazole

To a solution of 0.46 g (1.7 mmol) 4-amino-6-bromo-2-(N,N-dimethylamino)-1-methyl-1H-benzimidazole and 0.3 g (2.2 mmol) 2,6-dimethyl-benzaldehyde in 10 ml dichloromethane and 2.5 ml acetic acid were added 0.6 g (2.8 mmol) sodium triacetoxyborohydride. After 1 h at ambient temperature, saturated aqueous sodium hydrogen carbonate was added and stirring was continued for 30 min. The organic layer was separated, dried over anhydrous magnesium sulphate and evaporated. Purification of the residue by column chromatography on silica gel using ethyl acetate and crystallization from ethyl acetate/n-heptane yielded 0.46 g (70%) of the title compound as a colourless solid (m.p. 157-158° C.).

### AAA. 1-Benzyloxymethyl-6-bromo-4-(2,6-dimethyl-benzylamino)-2-methyl-1H-benzimidazole

To a suspension of 7.6 g (22 mmol) 4-amino-1-benzyloxymethyl-6-bromo-2-methyl-1H-benzimidazole and 3.56 g (23.0 mmol) 2,6-dimethyl-benzyl chloride in 140 ml acetonitrile were added 3.5 g (32.7 mmol) sodium carbonate and 0.66 g (4.4 mmol) sodium iodide. After 3.5 h reflux, the reaction mixture was partitioned between water and dichloromethane. The organic layer was separated, dried over anhydrous magnesium sulphate and evaporated. Purification of the residue by column chromatography on silica gel using ethyl acetate:light petroleum ether (2:3) and crystallization from ethyl acetate/n-heptane yielded 6.8 g (64%) of the title compound as a colourless solid (m.p. 147-148° C.).

II. Final Products of the Formula 1

### 1. Methyl 4-(2-ethyl-6-methyl-benzyloxy)-1,2-dimethyl-1H-benzimidazole-6-carboxylate

To a solution of 2.0 g (9.1 mmol) methyl 4-hydroxy-1,2-dimethyl-1H-benzimidazole carboxylate and 1.7 g (10.1 mmol) 2-ethyl-6-methyl-benzyl chloride in 56 ml N,N-dimethylformamide were slowly added 0.7 g (17.5 mmol) sodium hydride (60% dispersion in mineral oil). After 1 h, a further amount of 0.3 g (1.8 mmol) 2-ethyl-6-methyl-benzyl chloride was added and the mixture was stirred for 4 h. The mixture was carefully hydrolyzed with saturated aqueous ammonium chloride and partitioned between dichloromethane and water. The organic layer was dried over anhydrous magnesium sulphate and evaporated. Purification of the residue by crystallization from water/acetone yielded 2.7 g (84%) of the title compound as a solid (m.p. 157° C.).

### 2. 4-(2-Ethyl-6-methyl-benzyloxy)-1,2-dimethyl-1H-benzimidazole-6-carboxylic Acid

To a suspension of 2.6 g (7.4 mmol) methyl 4-(2-ethyl-6-methyl-benzyloxy)-1,2-dimethyl-1H-benzimidazole-6-carboxylate in 75 ml dioxane were added 15 ml 2N aqueous sodium hydroxide. After 3 h at 80° C., the reaction mixture was cooled down and the pH was adjusted to pH=7 by adding 6N hydrochloric acid. After addition of 50 g silica gel, the mixture was evaporated to dryness and the residue was purified by column chromatography on silica gel using dichloromethane:methanol (4:1) Evaporation of the solvent left a solid, which was crystallized from diethyl ether to give 2.67 g (quant.) of the title compound (crude product, contained silica gel) which was used without further purification for the next step.

### 3. Methyl 4-(2,6-dimethyl-benzyloxy)-1,2-dimethyl-1H-benzimidazole-6-carboxylate

To a solution of 3.6 g (16.4 mmol) methyl 4-hydroxy-1,2-dimethyl-1H-benzimidazole-6-carboxylate and 2.8 g (18.1 mmol) 2,6-dimethyl-benzyl chloride in 100 ml N,N-dimethylformamide were slowly added 1.3 g (32.5 mmol) sodium hydride (60% dispersion in mineral oil) over a period of 2 h. After complete reaction, the mixture was carefully hydrolyzed with saturated aqueous ammonium chloride and diluted with 500 ml water. The precipitate was collected, washed thoroughly with water and dried over phosphorus pentoxide to give 4.04 g (73%) of the title compound (m.p. 165-169° C.).

### 4. 4-(2,6-Dimethyl-benzyloxy)-1,2-dimethyl-1H-benzimidazole-6-carboxylic Acid

To a suspension of 3.5 g (10.3 mmol) methyl 4-(2,6-dimethyl-benzyloxy)-1,2-dimethyl-1H-benzimidazole-6-carboxylate in 100 ml dioxane were added 20 ml 2N aqueous sodium hydroxide. After 2 h at 80° C., the reaction mixture was cooled down and the pH was adjusted to pH=7 by adding 6N hydrochloric acid. After addition of 50 g silica gel, the mixture was evaporated to dryness and the residue was put on a column and eluted with dichloromethane:methanol (4:1). Evaporation of the solvent left a solid, which was crystallized from ethyl acetate/diethyl ether to give 2.33 g (70%) of the title compound (m.p. 285-286° C.).

### 5. 4-Benzyloxy-6-hydroxymethyl-1,2-dimethyl-1H-benzimidazole

To a suspension of 0.7 g (18.4 mmol) lithium aluminium hydride in 40 ml tetrahydrofuran was slowly added a solution of 3 g (9.7 mmol) methyl 4-benzyloxy-1,2-dimethyl-1H-benzimidazole-6-carboxylate in 10 ml tetrahydrofuran. After complete addition, the reaction mixture was carefully hydrolyzed with 0.13 ml water, 0.25 ml 6N aqueous potassium hydroxide and 0.13 ml water. Anhydrous magnesium sulphate was added and the mixture was stirred 1 h. After filtration of the suspension through celite, the filtrate was evaporated and the residue was crystallized from acetone to yield 1.99 g (74%) of the title compound as a colourless solid (m.p. 213-214° C.).

### 6. 4-Benzyloxy-6-methoxymethyl-1,2-dimethyl-1H-benzimidazole

To a solution of 1.5 g (5.3 mmol) 4-benzyloxy-6-hydroxymethyl-1,2-dimethyl-1H-benzimidazole in 12 ml N,N-dimethylformamide were slowly added 0.4 g (10 mmol) sodium hydride (60% dispersion in mineral oil) and the mixture was warmed to 50° C. After 1 h, the reaction mixture was cooled to −10° C. and 0.4 ml (6.4 mmol) methyl iodide were added over a period of 30 min. The reaction mixture was stirred 3 h and then carefully hydrolyzed with saturated aqueous ammonium chloride. The mixture was partitioned between dichloromethane and water. The organic layer was separated, dried over anhydrous magnesium sulphate and evaporated. Purification of the residue by crystallization from light petroleum ether yielded 1.25 g (80%) of the title compound as a solid (m.p. 113° C.).

### 7. 4-Benzyloxy-6-(N,N-dimethylaminocarbonyl)-1,2-dimethyl-1H-benzimidazole

To a solution of 3.0 g (9.1 mmol) 4-benzyloxy-6-bromo-1,2-dimethyl-1H-benzimidazole in 100 ml dimethylamine (3.2M in tetrahydrofuran) were added 0.3 g (1.3 mmol) palladium(II) acetate and 1.4 g (5.3 mmol) triphenylphosphine. The mixture was transferred to an autoclave and carbonylated (6 bar carbon monoxide pressure, 120° C.) for 16 h. The reaction mixture was cooled down, evaporated and the residue was dissolved in dichloromethane. The organic layer was washed with water, dried over anhydrous magnesium sulphate and evaporated. Purification of the residue by column chromatography on silica gel using ethyl acetate yielded 2.3 g (78%) of the title compound as a colourless solid (m.p. 159-160° C.).

### 8. 6-(N,N-Dimethylaminocarbonyl)-4-(2-ethyl-6-methyl-benzyloxy)-1,2-dimethyl-1H-benzimidazole

To a suspension of 0.35 g (1.5 mmol) 6-(N,N-dimethylaminocarbonyl)-4-hydroxy-1,2-dimethyl-1H-benzimidazole and 0.32 g (3 mmol) sodium carbonate in 5 ml acetone were added 0.5 g (3 mmol) 2-ethyl-6-methyl-benzyl chloride and the mixture was stirred 20 h at ambient temperature. The mixture was partitioned between dichloromethane and water. The organic layer was separated, dried over anhydrous magnesium sulphate and evaporated. Purification of the residue by column chromatography on silica gel using ethyl acetate yielded 0.38 g (68%) of the title compound as a colourless solid (m.p. 161-162° C.).

### 9. 6-(N,N-Dimethylaminocarbonyl)-4-(2,6-dimethyl-benzyloxy)-1,2-dimethyl-1H-benzimidazole

To a suspension of 1.0 g (3.08 mmol) 4-(2,6-dimethyl-benzyloxy)-1,2-dimethyl-1H-benzimidazole-6-carboxylic acid in 40 ml dichloromethane and 10 ml N,N-dimethylformamide were added 1.7 g (5.3 mmol) O-(1H-benzotriazol-1-yl)-N,N,N′,N′-tetramethyl-uronium tetrafluoroborate (TBTU) and the mixture was heated to 40° C. After 1 h, 3.7 ml (18.5 mmol) of dimethylamine (5M in tetrahydrofuran) were added at ambient temperature. After 30 min, the reaction mixture was partitioned between 2N aqueous sodium hydroxide and dichloromethane. The organic layer was separated, dried over anhydrous magnesium sulphate and evaporated. Purification of the residue by column chromatography on silica gel using dichloromethane:methanol (13:1) gave an oil which was crystallized from ethyl acetate/light petroleum ether to yield 1.0 g (91%) of the title compound as a yellow solid (m.p. 180° C.).

### 10. 4-(trans-2,3-Dihydro-2-hydroxy-1-indenyloxy)-6-(N,N-dimethylaminocarbonyl)-1,2-dimethyl-1H-benzimidazole Oxalate

To a suspension of 0.5 g (2.1 mmol) 6-(N,N-dimethylaminocarbonyl)-4-hydroxy-1,2-dimethyl-1H-benzimidazole and 1.1 g (8.3 mmol) 1,2-epoxyindane in 5.3 ml methanol and 1.3 ml water were added 0.6 ml triethylamine and the mixture was heated to 70° C. for 30 min. The cooled solution was partioned between dichloromethane and water. The organic layer was separated, dried over anhydrous magnesium sulphate and evaporated. Purification of the residue by column chromatography on silica gel using dichloromethane:methanol (13:1) gave an oil which was dissolved in acetone and treated with a solution of 0.27 g (2.1 mmol) oxalic acid dihydrate in acetone. The precipitate was collected and washed with acetone to yield 0.64 g (65%) of the title compound as a colourless solid (m.p. 144-145° C.).

### 11. 4-[(1S,2S)-2,3-Dihydro-2-hydroxy-1-indenyloxy]-6-(N,N-dimethylaminocarbonyl)-1,2-dimethyl-1H-benzimidazole Oxalate

To a suspension of 0.5 g (2.1 mmol) 6-N,N-dimethylaminocarbonyl)-4-hydroxy-1,2-dimethyl-1H-benzimidazole and 0.75 g (5.7 mmol) (1R,2S)-epoxyindane in 5 ml ethanol and 1.25 ml water were added 0.6 ml triethylamine and the mixture was heated to 60° C. for 48 h. The cooled solution was partitioned between dichloromethane and water. The organic layer was separated, dried over anhydrous magnesium sulphate and evaporated. Purification of the residue by column chromatography on silica gel using dichloromethane:methanol (13:1) gave an oil which was dissolved in acetone and treated with a solution of 0.12 g (1 mmol) oxalic acid dihydrate in acetone. The precipitate was collected and washed with acetone and diethylether to yield 0.14 g (14%) of the title compound as a colourless solid (m.p. 126-127° C., 92% ee).

### 12. 4-(trans-2,3-Dihydro-2-methoxy-1-indenyloxy)-6-(N,N-dimethylaminocarbonyl)-1,2-dimethyl-1H-benzimidazole Oxalate

To a solution of 0.57 g (1.56 mmol) 4-(trans-2,3-dihydro-2-hydroxy-1-indenyloxy)-6-(N,N-dimethylaminocarbonyl)-1,2-dimethyl-1H-benzimidazole in 5 ml N,N-dimethylformamide were slowly added 0.15 g (2.3 mmol) sodium hydride (60% dispersion in mineral oil) at 0° C. After 5 min, 0.13 m mmol) methyl iodide were added and stirring was continued for 40 min at ambient temperature. The reaction mixture was carefully hydrolyzed with 1 ml 1 N aqueous ammonia and partitioned between dichloromethane and water. The organic layer was separated, dried over anhydrous magnesium sulphate and evaporated. Purification of the residue by column chromatography on silica gel using toluene:dioxane:methanol (6:3:1) gave an oil which was dissolved in acetone and treated with a solution of 0.19 g (1.6 mmol) oxalic acid dihydrate in acetone. The precipitate was collected and washed with acetone and diethyl ether to yield 0.51 g (70%) of the title compound as a colourless solid (m.p. 126-127° C.).

### 13. 4-[trans-2,3-Dihydro-2-methoxymethylcarbonyloxy)-1-indenyloxy]-6-(N,N-dimethylaminocarbonyl)-1,2-dimethyl-1H-benzimidazole

To a solution of 0.2 ml (1.9 mmol) methoxyacetic acic in 4 ml tetrahydrofuran were added 0.3 g (1.9 mmol) N,N′-carbonyldiimidazole. After 15 min, 0.4 g (1.1 mmol) 4-trans-2,3-dihydro-2-hydroxy-1-indenyloxy)-6-(N,N-dimethylaminocarbonyl)-1,2-dimethyl-1H-benzimidazole and 0.3 ml (2 mmol) 1,8-diazabicyclo[5.4.0]undec-7-ene were added and stirring was continued for 1 h. The mixture was partitioned between dichloromethane and saturated aqueous ammonium chloride. The organic layer was separated, dried over anhydrous magnesium sulphate and evaporated. Purification of the residue by column chromatography on silica gel using toluene:dioxane:methanol (6:3.5:0.5) and crystallization from 2-propanol/diethyl ether/n-heptane yielded 0.47 g (97%) of the title compound as a solid (m.p. 140° C.).

### 14. 4-(trans-2,3-Dihydro-2-hydroxy-1-indenyloxy)-6-methoxymethyl-1,2-dimethyl-1H-benzimidazole Oxalate

To a suspension of 0.8 g (3.9 mmol) 4-hydroxy-methoxymethyl-1,2-dimethyl-1H-benzimidazole and 1.03 g (7.8 mmol) 1,2-epoxyindane in 9.5 ml methanol and 2.5 ml water were added 1.08 ml triethylamine and the mixture was heated to 50° C. for 4 h. The cooled solution was partitioned between dichloromethane and saturated aqueous ammonium chloride. The organic layer was separated, dried over anhydrous magnesium sulphate and evaporated. Purification of the residue by column chromatography on silica gel using dichloromethane:methanol (20:1) gave an oil which was dissolved in acetone and treated with a solution of 0.51 g (4 mmol) oxalic acid dihydrate in acetone. The precipitate was collected and washed with acetone to yield 0.72 g (42%) of the title compound as a colourless solid (purity: 93%).

### 15. Ethyl 4-(trans-2,3-dihydro-2-hydroxy-1-indenyloxy)-1,2-dimethyl-1H-benzimidazole-6-carboxylate

To a suspension of 5.0 g (21.4 mmol) ethyl 4-hydroxy-1,2-dimethyl-1H-benzimidazole-6-carboxylate and 5.65 g (42.8 mmol) 1,2-epoxyindane in 48 ml ethanol and 12 ml water were added 6 ml triethylamine and the mixture was heated to 60° C. for 4 h. The cooled solution was partitioned between dichloromethane and saturated aqueous ammonium chloride. The organic layer was separated, dried over anhydrous magnesium sulphate and evaporated. Purification of the residue by crystallization from ethanol/diethyl ether yielded 5.66 g (72%) of the title compound which was used without further purification in the next step.

### 16. 4-(trans-2,3-Dihydro-2-hydroxy-1-indenyloxy)-1,2-dimethyl-1H-benzimidazole-6-carboxylic Acid

To a suspension of 5.6 g (15.3 mmol) ethyl 4-(trans-2,3-dihydro-2-hydroxy-1-indenyloxy)-1,2-dimethyl 1H-benzimidazole-6-carboxylate in 50 ml dioxane were added 10 ml 2N aqueous sodium hydroxide. After 2.5 h at 80° C., the reaction mixture was cooled down and the pH was adjusted to pH=6 by adding 6N hydrochloric acid. After addition of 50 g silica gel, the mixture was evaporated to dryness and the residue was put on a column and eluted with dichloromethane:methanol (4:1). Evaporation of the solvent left a solid, which was crystallized from ethyl acetate/n-heptane to give 5.3 g (quant.) of the title compound (m.p. 306° C.).

### 17. Ethyl 4-(trans-5-chloro-2,3-dihydro-2-hydroxy-1-indenyloxy)-1,2-dimethyl-1H-benzimidazole-6-carboxylate

To a suspension of 1.17 g (5.0 mmol) ethyl 4-hydroxy-1,2-dimethyl-1H-benzimidazole-6-carboxylate and 2.47 g (10 mmol) 2-bromo-5-chloro-2,3-dihydro-1H-inden-1-ol in 20 ml ethanol and 5 ml water were added 4.14 g (30 mmol) potassium carbonate and the mixture was heated to 50° C. After 2 h, a further amount of 1 g (4 mmol) 2-bromo-5-chloro-2,3-dihydro-1H-inden-1-ol and 2 g (14.5 mmol) potassium carbonate were added and stirring was continued for 5 h. The cooled solution was partitioned between dichloromethane and saturated aqueous ammonium chloride. The organic layer was separated, dried over anhydrous magnesium sulphate and evaporated. Purification of the residue by column chromatography on silica gel using dichloromethane:methanol (100:3) yielded 0.52 g (26%) of the title compound which was used without further purification in the next step.

### 18. 4-(trans-5-Chloro-2,3-dihydro-2-hydroxy-1-indenyloxy)-1,2-dimethyl-1H-benzimidazole-6-carboxylic Acid

To a suspension of 0.51 g (1.27 mmol) ethyl 4-(trans-5-chloro-2,3-dihydro-2-hydroxy-1-indenyloxy)-1,2-dimethyl-1H-benzimidazole-6-carboxylate in 13 ml dioxane were added 2.5 ml 2N aqueous sodium hydroxide. After 4 h at 80° C., the reaction mixture was cooled down and the pH was adjusted to pH=6 by adding 6N hydrochloric acid. After addition of 50 g silica gel, the mixture was evaporated to dryness and the residue was put on a column and eluted with dichloromethane:methanol (4:1). Evaporation of the solvent left a solid, which was crystallized from n-heptane to give 0.63 g of the title compound (contained silica gel), which was used without further purification in the next step.

### 19. 4-(trans-5-Chloro-2,3-dihydro-2-hydroxy-1-indenyloxy)-6-(N,N-dimethylaminocarbonyl)-1,2-dimethyl-1H-benzimidazole Oxalate

To a suspension of 0.63 g (‘1.27 mmol’, contained silica gel) 4-(trans-5-chloro-2,3-dihydro-2-hydroxy-1-indenyloxy)-1,2-dimethyl-1H-benzimidazole-carboxylic acid in 14 ml dichloromethane and 7 ml N,N-dimethylformamide were added 0.6 g (1.9 mmol) O-(1H-benzotriazol-1-yl)-N,N,N′,N′-tetramethyl-uronium tetrafluoroborate (TBTU) and the mixture was heated to 40° C. After 30 min, 1.5 ml (7.5 mmol) dimethylamine (5M in tetrahydrofuran) were added at ambient temperature. After 30 min, the reaction mixture was partitioned between 2N aqueous sodium hydroxide and dichloromethane. The organic layer was separated, dried over anhydrous magnesium sulphate and evaporated. The residue was purified by column chromatography on silica gel using dichloromethane:methanol (13:1). The product fractions were evaporated and the residue was dissolved in acetone and treated with a solution of 0.27 g (2.1 mmol) oxalic acid dihydrate in acetone. After cooling, the precipitate was collected and washed with acetone to yield 0.42 g of the title compound as a colourless solid (m.p. 158-159° C.).

### 20. Ethyl 4-(trans-2,3-dihydro-2-hydroxy-4,7-dimethyl-1-indenyloxy)-1,2-dimethyl-1H-benzimidazole-6-carboxylate

To a suspension of 1.17 g (5.0 mmol) ethyl 4-hydroxy-1,2-dimethyl-1H-benzimidazole-6-carboxylate and 2.41 g (10 mmol) 2-bromo-2,3-dihydro-4,7-dimethyl-1H-inden-1-ol in 20 ml ethanol and 5 ml water were added 4.14 g (30 mmol) potassium carbonate and the mixture was heated to 50° C. After 1 h, a further amount of 2.41 g (10 mmol) 2-bromo-2,3-dihydro-4,7-dimethyl-1H-inden-1-ol and 1.4 g (10 mmol) potassium carbonate were added and stirring was continued 1 h. The cooled solution was partitioned between dichloromethane and saturated aqueous ammonium chloride. The organic layer was separated, dried over anhydrous magnesium sulphate and evaporated. Purification of the residue by column chromatography on silica gel using dichloromethane:methanol (100:3) and crystallization from ethyl acetate/n-heptane yielded 0.85 g (43%) of the title compound as a colourless solid (m.p. 184° C.).

### 21. 4-(trans-2,3-Dihydro-2-hydroxy-4,7-dimethyl-1-indenyloxy)-1,2-dimethyl-1H-benzimidazole-6-carboxylic Acid

To a suspension of 0.8 g (2 mmol) ethyl 4-(trans-2,3-dihydro-2-hydroxy-4,7-dimethyl-1-indenyloxy)-1,2-dimethyl-1H-benzimidazole-6-carboxylate in 20 ml dioxane were added 4 ml 2N aqueous sodium hydroxide. After 4 h at 80° C., the reaction mixture was cooled down and the pH was adjusted to pH=6 by adding 10% hydrochloric acid. After addition of 50 g silica gel, the mixture was evaporated to dryness and the residue was put on a column and eluted with dichloromethane:methanol (4:1). Evaporation of the solvent left 0.8 g of the title compound (contained silica gel) as a solid, which was used without further purification in the next step.

### 22. 4-(trans-2,3-Dihydro-2-hydroxy-4,7-dimethyl-1-indenyloxy)-6-(N,N-dimethylaminocarbonyl)-1,2-dimethyl-1H-benzimidazole

To a suspension of 0.7 g (‘1.8 mmol’, contained silica gel) 4-(trans-2,3-dihydro-2-hydroxy-4,7-dimethyl-1-indenyloxy)-1,2-dimethyl-1H-benzimidazole-6-carboxylic acid in 20 ml dichloromethane and 10 ml N,N-dimethylformamide were added 0.8 g (2.5 mmol) O-(1H-benzotriazol-1-yl)-N,N,N′,N′-tetramethyl-uronium tetrafluoroborate (TBTU) and the mixture was heated to 40° C. After 30 min, 2 ml (10 mmol) dimethylamine (5M in tetrahydrofuran) were added at ambient temperature. After 20 min, the reaction mixture was partitioned between 2N aqueous sodium hydroxide and dichloromethane. The organic layer was separated, dried over anhydrous magnesium sulphate and evaporated. The residue was purified by column chromatography on silica gel using dichloromethane:methanol (13:1). Crystallization from ethyl acetate/n-heptane yielded 0.72 g of the title compound as a colourless solid (m.p. 134° C.).

### 23. 4-(trans-2,3-Dihydro-2-hydroxy-1-indenyloxy)-1,2-dimethyl-6-[(1-pyrrolidino)carbonyl]-1H-benzimidazole Oxalate

To a suspension of 1.0 g (2.96 mmol) 4-trans-2,3-dihydro-2-hydroxy-1-indenyloxy)-1,2-dimethyl-1H-benzimidazole-6-carboxylic acid in 20 ml dichloromethane and 10 ml 1-methyl-2-pyrrolidinone were added 1.45 g (4.5 mmol) O-(1H-benzotriazol-1-yl)-N,N,N′,N′-tetramethyl-uronium tetrafluoroborate (TBTU) and the mixture was heated to 40° C. After 15 min, 0.49 ml (6 mmol) pyrrolidine were added at ambient temperature. After 2 h, the reaction mixture was partitioned between saturated aqueous ammonium chloride and dichloromethane. The organic layer was separated, dried over anhydrous magnesium sulphate and evaporated. The residue was purified by column chromatography on silica gel using dichloromethane:methanol (13:1). The oil thus obtained was dissolved in acetone and treated with a solution of 0.38 g (3 mmol) oxalic acid dihydrate in acetone. The precipitate was collected and washed with acetone and diethyl ether to yield 1.06 g (74%) of the title compound as a colourless solid (m.p. 124° C.).

### 24. 4-(trans-2,3-Dihydro-2-hydroxy-1-indenyloxy)-6-[N-(2-methoxyethyl)-N-ethyl-amino-carbonyl]-1,2-dimethyl-1H-benzimidazole Oxalate

To a suspension of 1.0 g (2.96 mmol) 4-(trans-2,3-dihydro-2-hydroxy-1-indenyloxy)-1,2-dimethyl-1H-benzimidazole-6-carboxylic acid in 20 ml dichloromethane and 10 ml 1-methyl-2-pyrrolidinone were added 1.45 g (4.5 mmol) O-(1H-benzotriazol-1-yl)-N,N,N′,N′-tetramethyl-uronium tetrafluoroborate (TBTU) and the mixture was heated to 40° C. After 15 min, 0.54 g (6 mmol) N-(2-methoxyethyl)-N-methyl-amine were added at ambient temperature. After 1 h, the reaction mixture was partitioned between saturated aqueous ammonium chloride and dichloromethane. The organic layer was separated, dried over anhydrous magnesium sulphate and evaporated. The residue was purified by column chromatography on silica gel using dichloromethane:methanol (13:1). The product fractions were evaporated and the residue was dissolved in acetone and treated with a solution of 0.19 g (1.5 mmol) oxalic acid dihydrate in acetone. The precipitate was collected and washed with acetone and diethyl ether to yield 0.55 g (38%) of the title compound as a colourless solid (m.p. 141° C.).

### 25. 4-(trans-2,3-Dihydro-2-hydroxy-1-indenyloxy)-1,2-dimethyl-6-[(1-piperidino)carbonyl]-1H-benzimidazole Oxalate

To a suspension of 1.3 g (3.84 mmol) 4-(trans-2,3-dihydro-2-hydroxy-1-indenyloxy)-1,2-dimethyl-1H-benzimidazole-6-carboxylic acid in 25 ml dichloromethane and 10 ml N,N-dimethylformamide were added 1.85 g (5.76 mmol) O-(1H-benzotriazol-1-yl)-N,N,N′,N′-tetramethyl-uronium tetrafluoroborate (TBTU) and the mixture was heated to 40° C. After 15 min, 0.64 ml (7.7 mmol) piperidine were added at ambient temperature. After 1 h, the reaction mixture was partitioned between saturated aqueous ammonium chloride and dichloromethane. The organic layer was separated, dried over anhydrous magnesium sulphate and evaporated. The residue was purified by column chromatography on silica gel using dichloromethane:methanol (13:1). The oil thus obtained was dissolved in acetone and treated with a solution of 0.25 g (2 mmol) oxalic acid dihydrate in acetone. The precipitate was collected and washed with acetone and diethyl ether to yield 0.95 g (50%) of the title compound as a colourless solid (m.p. 115-116° C.).

### 26. 6-(Cyclopropylaminocarbonyl)-4-(trans-2,3-dihydro-2-hydroxy-1-indenyloxy)-1,2-dimethyl-1H-benzimidazole

To a suspension of 1.0 g (2.96 mmol) 4-trans-2,3-dihydro-2-hydroxy-1-indenyloxy)-1,2-dimethyl-1H-benzimidazole-6-carboxylic acid in 20 ml dichloromethane and 10 ml N,N-dimethylformamide were added 1.45 g (4.5 mmol) O-(1H-benzotriazol-1-yl)-N,N,N′,N′-tetramethyl-uronium tetrafluoroborate (TBTU) and the mixture was heated to 40° C. After 1 h, 0.45 ml (6.5 mmol) cyclopropylamine were added at ambient temperature. After 1 h, the reaction mixture was partitioned between 2N aqueous sodium hydroxide and dichloromethane. The organic layer was separated, dried over anhydrous magnesium sulphate and evaporated. The residue was purified by crystallization from ethyl acetate/methanol/n-heptane to yield 0.78 g (70%) of the title compound as a colourless solid (m.p. 148-149° C.).

### 27. Ethyl 4-(2-ethyl-6-methyl-benzylamino)-1,2-dimethyl-1H-benzimidazole-6-carboxylate

1.0 g (2.7 mmol) 6-bromo-4-(2-ethyl-6-methyl-benzylamino)-1,2-dimethyl-1H-benzimidazole were d solved in 15 ml ethanol and 2.5 ml triethylamine and transferred to an autoclave. After addition of 0.1 g (0.45 mmol) palladium(II) acetate and 0.33 g (1.25 mmol) triphenylphosphine, the reaction mixture was carbonylated (5 bar carbon monoxide pressure, 100° C.) for 16 h. The reaction mixture was cooled down, filtered and evaporated to leave an orange coloured oil which was dissolved in ethyl acetate and extracted with water. The organic layer was separated, dried over anhydrous magnesium sulphate and evaporated. Purification of the residue by crystallization from ethyl acetate/light petroleum ether yielded 0.8 g (81%) of the title compound as a colourless solid (m.p. 171-173° C.).

### 28. Ethyl 4-(2,6-dimethyl-benzylamino)-1,2-dimethyl-1H-benzimidazole-6-carboxylate

8.8 g (24.6 mmol) 6-bromo-4-(2,6-dimethyl-benzylamino)-1,2-dimethyl-1H-benzimidazole were dissolved in 130 ml ethanol and 21.3 ml triethylamine and transferred to an autoclave. After addition of 0.8 g (3.6 mmol) palladium(II) acetate and 3.2 g (12.2 mmol) triphenylphosphine, the reaction mixture was carbonylated (10 bar carbon monoxide pressure, 100° C.) for 16 h. The reaction mixture was cooled down, filtered and evaporated to leave an orange coloured oil which was dissolved in dichloromethane and extracted with water. The organic layer was separated, dried over anhydrous magnesium sulphate and evaporated. Purification of the residue by column chromatography on silica gel using ethyl acetate:light petroleum ether (1:1) and crystallization from ethyl acetate yielded 6.18 g (72%) of the title compound as a colourless solid (m.p. 190° C.).

### 29. Ethyl 4-(2-ethyl-6-methyl-benzylamino)-2-methoxymethyl-1-methyl-1H-benzimidazole-6-carboxylate

3.4 g (8.45 mmol) 6-bromo-4-(2-ethyl-6-methyl-benzylamino)-2-methoxymethyl-1-methyl-1H-benzimidazole were dissolved in 80 ml ethanol and 10 ml triethylamine and transferred to an autoclave. After addition of 0.28 g (1.3 mmol) palladium(II) acetate and 1.1 g (4.2 mmol) triphenylphosphine, the reaction mixture was carbonylated (6 bar carbon monoxide pressure, 100° C.) for 16 h. The reaction mixture was cooled down, poured into 250 ml water and extracted with dichloromethane. The organic layer was separated, dried over anhydrous magnesium sulphate and evaporated. Purification of the residue by column chromatography on silica gel using ethyl acetate:light petroleum ether (3:7) and crystallization from diisopropyl ether yielded 2.2 g (66%) of the title compound as a colourless solid (m.p. 106-107° C.).

### 30. Ethyl 4-(2,6-dimethyl-benzylamino)-2-methoxymethyl-1-methyl-1H-benzimidazole-6-carboxylate

2.5 g (6.43 mmol) 6-bromo-4-(2,6-dimethyl-benzylamino)-2-methoxymethyl-1-methyl-1H-benzimidazole were dissolved in 60 ml ethanol and 7 ml triethylamine and transferred to an autoclave. After addition of 0.22 g (0.96 mmol) palladium(II) acetate and 0.84 g (3.2 mmol) triphenylphosphine, the reaction mixture was carbonylated (6 bar carbon monoxide pressure, 110° C.) for 16 h. The reaction mixture was cooled down, poured into 250 ml water and extracted with dichloromethane. The organic layer was separated, dried over anhydrous magnesium sulphate and evaporated. Purification of the residue by column chromatography on silica gel using ethyl acetate:light petroleum ether (1:1) and crystallization from diisopropyl ether yielded 1.84 g (81%) of the title compound as a yellow solid (m.p. 134-135° C.).

### 31. 4-(2-Ethyl-6-methyl-benzylamino)-1,2-dimethyl-1H-benzimidazole-6-carboxylic Acid

To a suspension of 5.0 g (13.7 mmol) ethyl 4-(2-ethyl-6-methyl-benzylamino)-1,2-dimethyl-1H-benzimidazole-6-carboxylate in 80 ml dioxane were added 50 ml 2N aqueous sodium hydroxide. After 2 h at 100° C., the reaction mixture was cooled down, poured into 50 ml saturated aqueous ammonium chloride and the pH was adjusted to pH=6 by adding 6N hydrochloric acid. The thick precipitate was collected, washed with water and recrystallized from ethanol to give 3.7 g (81%) of the title compound as a colourless solid (m.p. 312-314° C.).

### 32. 4-(2,6-Dimethyl-benzylamino)-1,2-dimethyl-1H-benzimidazole-6-carboxylic Acid

To a suspension of 5.0 g (14.2 mmol) ethyl 4-(2,6-dimethyl-benzylamino)-1,2-dimethyl-1H-benzimidazole-6-carboxylate in 50 ml dioxane were added 20 ml 2N aqueous sodium hydroxide. After 16 h at 100° C., the reaction mixture was cooled down and the pH was adjusted to pH=7 by adding 6N hydrochloric acid. After addition of 50 g silica gel, the mixture was evaporated to dryness and the residue was put on a column and eluted with dichloromethane:methanol (13:1). Evaporation of the solvent left a solid, which was crystallized from acetone to give 4.15 g (90%) of the title compound (m.p. 315-318° C.).

### 33. 4-(2-Ethyl-6-methyl-benzylamino)-2-methoxymethyl-1-methyl-1H-benzimidazole-6-carboxylic Acid

To a suspension of 1.6 g (4.04 mmol) ethyl 4-(2-ethyl-6-methyl-benzylamino)-2-methoxymethyl-1-methyl-1H-benzimidazole-6-carboxylate in 30 ml dioxane were added 16 ml 2N aqueous sodium hydroxide. After 2 h at 100° C., the reaction mixture was cooled down, poured into 30 ml water and the pH was adjusted to pH=6 by adding 6N hydrochloric acid. The precipitate was collected, washed with water and ethanol and dried over phosphorus pentoxide to give 1.06 g (71%) of the title compound as a colourless solid (m.p. 275-278° C.).

### 34. 4-(2,6-Dimethyl-benzylamino)-2-methoxymethyl-1-methyl-1H-benzimidazole-6-carboxylic Acid

To a suspension of 1.3 g (3.4 mmol) ethyl 4-(2,6-dimethyl-benzylamino)-2-methoxymethyl-1-methyl-1H-benzimidazole-6-carboxylate in 25 ml dioxane were added 13 ml 2N aqueous sodium hydroxide. After 3 h at 100° C., the reaction mixture was cooled down, poured into 75 ml water and the pH was adjusted to pH=6 by adding 6N hydrochloric acid. The precipitate was collected, washed with water, ethanol and diisopropyl ether to give 0.96 g (80%) of the title compound as a colourless solid (m.p. 269-272° C.).

### 35. 4-(2-Ethyl-6-methyl-benzylamino)-6-(2-hydroxyethyl-aminocarbonyl)-1,2-dimethyl-1H-benzimidazole

A suspension of 1.0 g (2.74 mmol) ethyl 4-(2-ethyl-6-methyl-benzylamino)-1,2-dimethyl-1H-benzimidazole-6-carboxylate in 10 ml 2-aminoethanol was heated to 110° C. for 30 h. The reaction mixture was cooled down, diluted with saturated aqueous ammonium chloride and extracted with ethyl acetate. The organic layer was separated, dried over anhydrous magnesium sulphate and evaporated. Purification of the residue by crystallization from ethyl acetate/diethyl ether yielded 0.72 g (69%) of the title compound as a colourless solid (m.p. 200-201° C.).

### 36. 4-(2,6-Dimethyl-benzylamino)-6-(2-hydroxyethyl-aminocarbonyl)-1,2-dimethyl-1H-benzimidazole

A suspension of 1.0 g (2.85 mmol) ethyl 4-(2,6-dimethyl-benzylamino)-1,2-dimethyl-1H-benzimidazole-6-carboxylate in 15 ml 2-aminoethanol was heated to 100° C. for 16 h. The reaction mixture was cooled down, diluted with water and extracted with dichloromethane/methanol. The organic layer was separated, dried over anhydrous magnesium sulphate and evaporated. Purification of the residue by crystallization from ethyl acetate/methanol and activated charcoal yielded 0.64 g (61%) of the title compound as a colourless solid (m.p. 259-260° C.).

### 37. 4-(2,6-Dimethyl-benzylamino)-6-(2-hydroxyethyl-aminocarbonyl)-2-methoxymethyl-1-methyl-1H-benzimidazole

A suspension of 0.4 g (1.04 mmol) ethyl 4-(2,6-dimethyl-benzylamino)-2-methoxymethyl-1-methyl-1H-benzimidazole-6-carboxylate in 4 ml 2-aminoethanol was heated to 140° C. for 3 h. The reaction mixture was cooled down, poured into 15 ml water and 10 ml saturated aqueous ammonium chloride and extracted with dichloromethane. The organic layer was separated, dried over anhydrous magnesium sulphate and evaporated. Purification of the residue by column chromatography on silica gel using toluene:dioxane:methanol (6:3.6:0.4) and crystallization from diisopropyl ether yielded 0.38 g (93%) of the title compound as a colourless solid (m.p. 186-187° C.).

### 38. 4-(2-Ethyl-6-methyl-benzylamino)-1,2-dimethyl-1H-benzimidazole-6-carboxamide

To a suspension of 1.0 g (2.96 mmol) 4-(2-ethyl-6-methyl-benzylamino)-1,2-dimethyl-1H-benzimidazole-6-carboxylic acid in 10 ml tetrahydrofuran and 5 ml N,N-dimethylformamide were added 0.54 g (3.26 mmol) N,N′-carbonyldiimidazole. After 1 h, 10 ml saturated methanolic ammonia were added and stirring was continued for 1 h. 25 ml saturated aqueous ammonium chloride and 50 ml water were added and the mixture was extracted with dichloromethane. The organic layer was separated, dried over anhydrous magnesium sulphate and evaporated. Purification of the residue by column chromatography on silica gel using toluene:dioxane:methanol (6:3.8:0.2) and crystallization from ethyl acetate/n-heptane yielded 0.8 g (80%) of the title compound as a colourless solid (m.p. 240-241° C.).

### 39. 4-(2,6-Dimethyl-benzylamino)-1,2-dimethyl-1H-benzimidazole-6-carboxamide

To a suspension of 1.2 g (3.7 mmol) 4-(2,6-dimethyl-benzylamino)-1,2-dimethyl-1H-benzimidazole-6-carboxylic acid in 10 ml tetrahydrofuran and 5 ml N,N-dimethylformamide were added 0.7 g (4.3 mmol) N,N′-carbonyldiimidazole. After 1 h, ammonia gas was bubbled through the flask for 1 h. The mixture was then partitioned between dichloromethane and 2N aqueous sodium hydroxide. The organic layer was separated, dried over anhydrous magnesium sulphate and evaporated. Purification of the residue by crystallization from ethyl acetate yielded 0.4 g (34%) of the title compound as a colourless solid (m.p. 272° C.).

### 40. 4-(2-Ethyl-6-methyl-benzylamino)-1,2-dimethyl-6-(pyrrolidinocarbonyl)-1H-benzimidazole

To a suspension of 1.0 g (2.96 mmol) 4-(2-ethyl-6-methyl-benzylamino)-1,2-dimethyl-1H-benzimidazole-carboxylic acid in 10 ml tetrahydrofuran and 3 ml N,N-dimethylformamide were added 0.54 g (3.26 mmol) N,N′-carbonyldiimidazole. After 2 h, 0.63 g (3.26 mmol) pyrrolidine were added and stirring was continued. After 1 h, 20 ml saturated aqueous ammonium chloride and 50 ml water were added and the mixture was extracted with dichloromethane. The organic layer was separated, dried over anhydrous magnesium sulphate and evaporated. Purification of the residue by column chromatography on silica gel using toluene:dioxane:methanol (6:3.9:0.1) and crystallization from ethyl acetate/n-heptane yielded 0.91 g (79%) of the title compound as a solid (m.p. 126° C.).

### 41. 4-(2-Ethyl-6-methyl-benzylamino)-1,2-dimethyl-6-(morpholinocarbonyl)-1H-benzimidazole

To a suspension of 1.5 g (4.44 mmol) 4-(2-ethyl-6-methyl-benzylamino)-1,2-dimethyl-1H-benzimidazole-6-carboxylic acid in 20 ml tetrahydrofuran and 5 ml N,N-dimethylformamide were added 1.48 g (8.88 mmol) N,N′-carbonyldiimidazole and the mixture was heated to 60° C. After 1 h, 1.94 g (22.2 mmol) morpholine were added and the reaction mixture was refluxed for 1 h. The mixture was then poured into 100 ml water and 20 ml saturated aqueous sodium hydrogen carbonate and extracted with dichloromethane. The organic layer was separated, dried over anhydrous magnesium sulphate and evaporated. Purification of the residue by column chromatography on silica gel using toluene:dioxane:methanol (6:3.9:0.1) and crystallization from diisopropyl ether/n-heptane yielded 1.6 g (89%) of the title compound as a colourless solid (m.p. 229-231° C.).

### 42. 6-(N,N-Dimethylaminocarbonyl)-4-(2-ethyl-6-methyl-benzylamino)-2-methoxymethyl-1-methyl-1H-benzimidazole

To a suspension of 0.9 g (2.44 mmol) 4-(2-ethyl-6-methyl-benzylamino)-2-methoxymethyl-1-methyl-1H-benzimidazole-carboxylic acid in 10 ml tetrahydrofuran and 3 ml N,N-dimethylformamide were added 0.8 g (4.9 mmol) N,N′-carbonyldiimidazole. After 1 h, 5 ml (5 mmol) dimethylamine (5M in tetrahydrofuran) were added and stirring was continued for 4 h. The mixture was poured into 60 ml water and extracted with dichloromethane. The organic layer was separated, washed with water, dried over anhydrous magnesium sulphate and evaporated. Purification of the residue by column chromatography on silica gel using toluene:dioxane:methanol (6:3.9:0.1) and crystallization from diisopropyl ether yielded 0.88 g (91%) of the title compound as a colourless solid (m.p. 154° C.).

### 43. 6-(N,N-Dimethylaminocarbonyl)-4-(2,6-dimethyl-benzylamino)-2-methoxymethyl-1-methyl-1H-benzimidazole

To a suspension of 0.5 g (1.4 mmol) 4-(2,6-dimethyl-benzylamino)-2-methoxymethyl-1-methyl-1H-benzimidazole-6-carboxylic acid in 8 ml tetrahydrofuran and 3 ml N,N-dimethylformamide were added 0.46 g (2.8 mmol) N,N′-carbonyldiimidazole and the mixture was heated to 60° C. for 5 min. After 1 h at ambient temperature, 3 ml (15 mmol) dimethylamine (5M in tetrahydrofuran) were added and stirring was continued for 1 h. The mixture was poured into 40 ml water and extracted with dichloromethane. The organic layer was separated, washed with water, dried over anhydrous magnesium sulphate and evaporated. Purification of the residue by column chromatography on silica gel using toluene:dioxane:methanol (6:3.8:0.2) and crystallization from diisopropyl ether yielded 0.49 g (92%) of the title compound as a colourless solid (m.p. 156-157° C.).

### 44. 6-N,N-Dimethylaminocarbonyl)-4-(2-ethyl-6-methyl-benzylamino)-1,2-dimethyl-1H-benzimidazole

To a solution of 9.5 g (25.5 mmol) 6-bromo-4-(2-ethyl-6-methyl-benzylamino)-1,2-dimethyl-1H-benzimidazole in 225 ml dimethylamine (3.2M in tetrahydrofuran) were added 0.85 g (2.55 mmol) palladium(II) acetate and 4 g (15.3 mmol) triphenylphosphine. The mixture was transferred to an autoclave and carbonylated (6 bar carbon monoxide pressure, 120° C.) for 16 h. The reaction mixture was cooled down, poured into 400 ml water and extracted with dichloromethane. The organic layer was separated, washed with water, dried over anhydrous magnesium sulphate and evaporated. Purification of the residue by column chromatography on silica gel using dichloromethane:methanol (100:3) and crystallization from boiling ethyl acetate yielded 5.8 g (76%) of the title compound as a colourless solid (m.p. 152-153° C.).

### 45. 6-(N,N-Dimethylaminocarbonyl)-4-(2-ethyl-6-methyl-benzylamino)-1-methyl-1H-benzimidazole Hydrochloride

To a solution of 2.0 g (5.6 mmol) 6-bromo-4-(2-ethyl-6-methyl-benzylamino)-1-methyl-1H-benzimidazole in 30 ml dimethylamine (5M in tetrahydrofuran) were added 0.2 g (0.9 mmol) palladium(II) acetate and 0.7 g (2.7 mmol) triphenylphosphine. The mixture was transferred to an autoclave and carbonylated (6 bar carbon monoxide pressure, 110° C.) for 16 h. The reaction mixture was cooled down, diluted with water and extracted with dichloromethane. The organic layer was separated, washed with water, dried over anhydrous magnesium sulphate and evaporated. Purification of the residue was achieved by column chromatography on silica gel using first dichloromethane:methanol (13:1), then ethyl acetate. The product fractions were evaporated and treated with saturated hydrogen chloride in diethyl ether to give 0.3 g (14%) of the title compound as a colourless solid (m.p. 183-184° C.).

### 46. 6-(N,N-Dimethylaminocarbonyl)-4-(2,6-dimethyl-benzylamino)-1,2-dimethyl-1H-benzimidazole

To a solution of 0.86 g (2.4 mmol) 6-bromo-4-(2,6-dimethyl-benzylamino)-1,2-dimethyl-1H-benzimidazole in 30 ml dimethylamine (5M in tetrahydrofuran) were added 80 mg (0.36 mmol) palladium(II) acetate and 250 mg (0.96 mmol) triphenylphosphine. The mixture was transferred to an autoclave and carbonylated (5.5 bar carbon monoxide pressure, 110° C.) for 16 h. The reaction mixture was cooled down, evaporated and partitioned between dichloromethane and water. The organic layer was separated, dried over anhydrous magnesium sulphate and evaporated. Purification of the residue by column chromatography on silica gel using dichloromethane:methanol (20:1) and crystallization from ethyl acetate/n-heptane yielded 0.4 g (48%) of the title compound as a colourless solid (m.p. 142-143° C.).

### 47. 6-(1-Azetidinocarbonyl)-4-(2,6-dimethyl-benzylamino)-1,2-dimethyl-1H-benzimidazole

To a suspension of 1.1 g (3.4 mmol) 4-(2,6-dimethyl-benzylamino)-1,2-dimethyl-1H-benzimidazole-6-carboxylic acid in 20 ml dichloromethane and 5 ml N,N-dimethylformamide were added 1.2 g (3.74 mmol) O-(1H-benzotriazol-1-yl)-N,N,N′,N′-tetramethyl-uronium tetrafluoroborate (TBTU) and the mixture was heated to 40° C. After 1 h, a solution of 0.36 g (3.74 mmol) azetidine hydrochloride in 1.14 g (11.2 mmol) triethylamine was added. After 45 min, the reaction mixture was poured into 120 ml water and extracted with dichloromethane. The organic layer was separated, dried over anhydrous magnesium sulphate and evaporated. The residue was purified by column chromatography on silica gel using toluene:dioxane:methanol (6:3.6:0.4). Crystallization from diisopropyl ether yielded 0.46 g (37%) of the title compound as a colourless solid (m.p. 193-195° C.).

### 48. 1-Benzyloxymethyl-6-(N,N-dimethylaminocarbonyl)-4-(2-ethyl-6-methyl-benzylamino)-2-methyl-1H-benzimidazole Oxalate

To a solution of 6.5 g (13.6 mmol) 1-benzyloxymethyl-6-bromo-4-(2-ethyl-4-methyl-benzylamino)-2-methyl-1H-benzimidazole in 44 ml dimethylamine (5M in tetrahydrofuran) were added 0.3 g (1.36 mmol) palladium(II) acetate and 2.1 g (8.1 mmol) triphenylphosphine. The mixture was transferred to an autoclave and carbonylated (6 bar carbon monoxide pressure, 100° C.) for 16 h. The reaction mixture was cooled down, poured into 200 ml water and extracted with ethyl acetate. The organic layer was separated, washed with water, dried over anhydrous magnesium sulphate and evaporated. Purification of the residue by column chromatography on silica gel using ethyl acetate:light petroleum ether (1:1) gave 3.1 g of a yellow oil, which was dissolved in 20 ml acetonitrile and treated with a solution of 0.8 g (6.4 mmol) oxalic acid dihydrate in 5 ml acetonitrile. The precipitate was collected and washed with diethyl ether to yield 1.34 g (18%) of the title compound as a solid (m.p. 168-169° C.).

### 49. 6-(N,N-Dimethylaminocarbonyl)-4-(2-ethyl-6-methyl-benzylamino)-2-methyl-1H-benzimidazole

0.7 g (1.25 mmol) 1-benzyloxymethyl-6-(N,N-dimethylaminocarbonyl)-4-(2-ethyl-6-methyl-benzylamino)-2-methyl-1H-benzimidazole oxalate were partitioned between dichloromethane and saturated aqueous sodium hydrogen carbonate. The organic layer was separated and evaporated. The residue was dissolved in 15 ml methanol and hydrogenated over 0.3 g 10% Pd/C (70° C., 1 bar H₂) for 2 h. The catalyst was filtered off and the filtrate was evaporated. The residue was purified by column chromatography on silica gel using first dichloromethane:methanol (13:1), then ethyl acetate:triethylamine (9:1). Crystallization from diisopropyl ether yielded 0.1 g (23%) of the title compound as a solid (m.p. 228-230° C.).

### 50. 6-(N,N-Dimethylaminocarbonyl)-4-(2-methoxycarbonylamino-6-methyl-benzylamino)-1,2-dimethyl-1H-benzimidazole

To a suspension of 0.38 g (1.63 mmol) 4-amino-6-(N,N-dimethylaminocarbonyl)-1,2-dimethyl-1H-benzimidazole and 0.37 g (1.71 mmol) 2-methoxycarbonylamino-6-methyl-benzyl chloride in 20 ml acetone were added 0.35 g (3.26 mmol) sodium carbonate and 25 mg (0.16 mmol) sodium iodide. After 3 h stirring at ambient temperature, the reaction mixture was poured into 80 ml water and extracted with dichloromethane. The organic layer was separated, dried over anhydrous magnesium sulphate and evaporated. Purification of the residue by column chromatography on silica gel using toluene:dioxane:methanol (6:3.8:0.2) and crystallization from diisopropyl ether yielded 0.37 g (55%) of the title compound as a colourless solid (m.p. 193-195° C.).

### 51. 4-(trans-2,3-Dihydro-2-hydroxy-1-indenylamino)-6-(N,N-dimethylaminocarbonyl)-1,2-dimethyl-1H-benzimidazole

To a suspension of 0.6 g (2.6 mmol) 4-amino-6-(N,N-dimethylaminocarbonyl)-1,2-dimethyl-1H-benzimidazole and 1 g (7.7 mmol) 1,2-epoxyindane in 10 ml dioxane and 2 ml water was added one drop of triethylamine and the reaction mixture was stirred at 100° C. for 6 h. After cooling down, the mixture was poured into 100 ml water and extracted with dichloromethane. The organic layer was separated, washed with water, dried over anhydrous magnesium sulphate and evaporated. Purification of the residue by column chromatography on silica gel using toluene:dioxane:methanol (6:3:1) and crystallization from diisopropyl ether yielded 0.72 g (77%) of the title compound as a colourless solid (m.p. 219-221° C.).

### 52. Ethyl 4-benzyloxy-1,2-dimethyl-1H-benzimidazole-6-carboxylate

To a solution of 15.0 g (45.3 mmol) 4-benzyloxy-6-bromo-1,2-dimethyl-1H-benzimidazole in 200 ml ethanol and 50 ml triethylamine were added 1.53 g (6.8 mmol) palladium(II) acetate and 5.35 g (20.4 mmol) triphenylphosphine. The mixture was transferred to an autoclave and carbonylated (10 bar carbon monoxide pressure, 100° C.) for 18 h. The reaction mixture was cooled down, evaporated and the residue was dissolved in dichloromethane. The organic layer was extracted with water, dried over anhydrous magnesium sulphate and evaporated. Purification of the residue by crystallization from ethyl acetate/n-heptane and activated charcoal yielded 12.3 g (84%) of the title compound as a colourless solid (m.p. 152° C.).

### 53. Methyl 4-benzyloxy-1,2-dimethyl-1H-benzimidazole-6-carboxylate

To a solution of 27.0 g (81 mmol) 4-benzyloxy-6-bromo-1,2-dimethyl-1H-benzimidazole in 340 ml methanol and 90 ml triethylamine were added 2.7 g (12 mmol) palladium(II) acetate and 9.6 g (37 mmol) triphenylphosphine. The mixture was transferred to an autoclave and carbonylated (10 bar carbon monoxide pressure, 100° C.) for 16 h. The reaction mixture was cooled down, filtered and evaporated. The residue was dissolved in dichloromethane, extracted with water, dried over anhydrous magnesium sulphate and evaporated. The residue was dissolved in hot ethyl acetate and treated with activated charcoal. After filtration, the filtrate was evaporated to leave a dark brown solid which was purified by column chromatography on silica gel using dichloromethane:methanol (30:1) to yield 15.6 g (62%) of the title compound as a grey solid (m.p. 166° C.).

### 54. 6-(1-Aziridinylcarbonyl)-4-(2,6-dimethyl-benzylamino)-1,2-dimethyl-1H-benzimidazole

To a suspension of 1.0 g (3.1 mmol) 4-(2,6-dimethyl-benzylamino)-1,2-dimethyl-1H-benzimidazole-6-carboxylic acid in 20 ml tetrahydrofuran and 10 ml N,N-dimethylformamide were added 0.9 g (5.6 mmol) N,N′-carbonyldiimidazole. After 2 h at 60° C., the solution was cooled to 30° C. and 1.5 ml (29 mmol) aziridine were added in three portions over a period of 1.5 h. 50 ml water were added and the mixture was extracted with dichloromethane. The organic layer was separated, dried over anhydrous magnesium sulphate and evaporated. Purification of the residue by column chromatography on silica gel using ethyl acetate and crystallization from ethyl acetate/n-heptane yielded 0.92 g (86%) of the title compound as a colourless solid (m.p. 201-202° C.).

### 55. 4-(2,6-Dimethyl-benzylamino)-1,2-dimethyl-6-(N-methylaminocarbonyl)-1H-benzimidazole

To a suspension of 1.0 g (3.1 mmol) 4-(2,6-dimethyl-benzylamino)-1,2-dimethyl-1H-benzimidazole-6-carboxylic acid in 20 ml tetrahydrofuran and 10 ml N,N-dimethylformamide were added 0.9 g (5.6 mmol) N,N′-carbonyldiimidazole. After 1.5 h at 60° C., the solution was cooled down to 40° C. and 3.05 ml (6.1 mmol) methylamine (2M in tetrahydrofuran) were added. After 30 min, saturated aqueous ammonium chloride was added and the mixture was extracted with dichloromethane. The organic layer was separated, dried over anhydrous magnesium sulphate and evaporated. Purification of the residue by crystallization from ethyl acetate yielded 1.0 g (96%) of the title compound as a colourless solid (m.p. 252-253° C.).

### 56. 4-(2,6-Dimethyl-benzylamino)-6-(N-2-hydroxyethyl-N-methylaminocarbonyl)-1,2-dimethyl-1H-benzimidazole

To a suspension of 1.0 g (3.1 mmol) 4-(2,6-dimethyl-benzylamino)-1,2-dimethyl-1H-benzimidazole-6-carboxylic acid in 45 ml dichloromethane and 10 ml N,N-dimethylformamide were added 1.9 g (5.9 mmol) O-(1H-benzotriazol-1-yl)-N,N,N′,N′-tetramethyl-uronium tetrafluoroborate (TBTU) and the mixture was heated to 40° C. After 1 h, 0.6 ml (8.0 mmol) 2-(methylamino)ethanol were added at ambient temperature. After 4 h, the reaction mixture was partitioned between 6N aqueous sodium hydroxide and dichloromethane. The organic layer was separated, dried over anhydrous magnesium sulphate and evaporated. The residue was purified by column chromatography on silica gel using dichloromethane:methanol (13:1). Crystallization from acetone/diethyl ether yielded 0.77 g (65%) of the title compound as a colourless solid (m.p. 177-178° C.).

### 57. 4-(2,6-Dimethyl-benzylamino)-1,2-dimethyl-1H-benzimidazole-6-carboxylic acid hydrazide

To a suspension of 1.0 g (3.1 mmol) 4-(2,6-dimethyl-benzylamino)-1,2-dimethyl-1H-benzimidazole-6-carboxylic acid in 20 ml tetrahydrofuran and 10 ml N,N-dimethylformamide were added 0.9 g (5.6 mmol) N,N′-carbonyldiimidazole. After 1.5 h at 60° C., the solution was cooled down to 40° C. and 0.3 ml (9.4 mmol) hydrazine were added. After 1 h, the precipitate was collected and recrystallized from 2-propanol to yield 0.88 g (85%) of the title compound as a colourless solid (m.p. 277-278° C.).

### 58. 4-(2,6-Dimethyl-benzylamino)-6-hydroxymethyl-1,2-dimethyl-1H-benzimidazole

To a suspension of 0.8 g (21 mmol) lithium aluminium hydride in 40 ml dried tetrahydrofuran was slowly added a solution of 4.0 g (11.4 mmol) ethyl 4-(2,6-dimethyl-benzylamino)-1,2-dimethyl-1H-benzimidazole-6-carboxylate in 20 ml dried tetrahydrofuran at 0° C. under a nitrogen atmosphere. After 2 h, an additional amount of 0.4 g (10.5 mmol) lithium aluminium hydride was added and stirring was continued at room temperature for 1 h. The reaction mixture was cautiously hydrolyzed with 0.3 ml water, 0.6 ml 6N aqueous potassium hydroxide and 0.3 ml water. Anhydrous magnesium sulphate was added, the suspension was filtered through celite and washed with boiling dichloromethane. After addition of 50 g silica gel, the mixture was evaporated to dryness and the residue was put on a column and eluted with dichloromethane:methanol (20:1). Evaporation of the solvent left a solid, which was crystallized from ethyl acetate/n-heptane to give 1.4 g (40%) of the title compound as a colourless solid (m.p. 245-246° C.).

### 59. 2-Cyclopropyl-4-(trans-2,3-dihydro-2-hydroxy-1-indenyloxy)-1-methyl-1H-benzimidazole-6-carboxylic acid

To a suspension of 1.5 g (5.8 mmol) ethyl 2-cyclopropyl-4-hydroxy-1-methyl-1H-benzimidazole-6-carboxylate and 1.5 g (11.5 mmol) 1,2-epoxyindane in 18 ml ethanol and 5 ml water were added 1.6 ml triethylamine and the mixture was heated to 50° C. for 4 h. The cooled solution was partitioned between dichloromethane and saturated aqueous ammonium chloride. The organic layer was separated, dried over anhydrous magnesium sulphate and evaporated. Purification of the residue by column chromatography on silica gel using dichloromethane:methanol (13:1) and crystallization from ethyl acetate/n-heptane yielded 0.8 g of a solid, which was suspended in 20 ml dioxane and 4 ml 2N aqueous sodium hydroxide. After 3 h at 80° C., the reaction mixture was cooled down and the pH was adjusted to pH=6 by adding 6N hydrochloric acid. After addition of 50 g silica gel, the mixture was evaporated to dryness and the residue was put on a column and eluted with dichloromethane:methanol (10:1). Evaporation of the solvent left a solid, which was crystallized from ethyl acetate/diethyl ether to give 0.68 g (32%) of the title compound (m.p. 132-134° C.).

### 60. 2-Cyclopropyl-4-(trans-2,3-dihydro-2-hydroxy-1-indenyloxy)-6-(N,N-dimethylaminocarbonyl)-1-methyl-1H-benzimidazole

To a suspension of 0.67 g (1.8 mmol) 2-cyclopropyl-4-(trans-2,3-dihydro-2-hydroxy-1-indenyloxy)-1-methyl-1H-benzimidazole-6-carboxylic acid in 30 ml dichloromethane and 7 ml N,N-dimethylformamide were added 1.0 g (3.1 mmol) O-(1H-benzotriazol-1-yl)-N,N,N′,N′-tetramethyl-uronium tetrafluoroborate (TBTU) and the mixture was heated to 40° C. After 1 h, 2.3 ml (11.4 mmol) dimethylamine (5M in tetrahydrofuran) were added at ambient temperature. After 30 min, the reaction mixture was partitioned between 2N aqueous sodium hydroxide and dichloromethane. The organic layer was separated, dried over anhydrous magnesium sulphate and evaporated. The residue was purified by column chromatography on silica gel using dichloromethane:methanol (13:1) to yield 0.63 g (87%) of a colourless foam.

### 61. 6-(N,N-Dimethylaminocarbonyl)-1,2-dimethyl-4-(2-methyl-benzylamino)-1H-benzimidazole

To a solution of 1.2 g (3.5 mmol) 6-bromo-1,2-dimethyl-4-(2-methyl-benzylamino)-1H-benzimidazole in 55 ml dimethylamine (5M in tetrahydrofuran) were added 0.24 g (0.3 mmol) bis(triphenylphosphine)palladium(II) chloride. The mixture was transferred to an autoclave and carbonylated (5 bar carbon monoxide pressure, 100° C.) for 16 h. The reaction mixture was cooled down and filtered. The filtrate was partitioned between dichloromethane and water. The organic layer was separated, dried over anhydrous magnesium sulphate and evaporated. Purification of the residue by column chromatography on silica gel using toluene:dioxane:methanol (6:3.9:0.1) and crystallization from ethyl acetate/n-heptane yielded 0.37 g (31%) of the title compound as a colourless solid (m.p. 158-159° C.).

### 62. 6-(N,N-Dimethylaminocarbonyl)-4-(2,4-dimethyl-furan-3-yl-methylamino)-1,2-dimethyl-1H-benzimidazole

To a solution of 0.4 g (1.15 mmol) 6-bromo-4-2,4-dimethyl-furan-3-yl-methylamino)-1,2-dimethyl-1H-benzimidazole in 20 ml dimethylamine (2M in tetrahydrofuran) were added 40 mg (0.17 mmol) palladium(II) acetate and 0.12 g (0.46 mmol) triphenylphosphine. The mixture was transferred to an autoclave and carbonylated (6 bar carbon monoxide pressure, 120° C.) for 16 h. The reaction mixture was cooled down and filtered. The filtrate was partitioned between dichloromethane and water. The organic layer was separated, dried over anhydrous magnesium sulphate and evaporated. Purification of the residue by column chromatography on silica gel using dichloromethane:methanol (20:1) and crystallization from ethyl acetate/n-heptane yielded 0.1 g (26%) of the title compound as a colourless solid (m.p. 163-164° C.).

### 63. 7-(2,6-Dimethyl-benzylamino)-2,3-dimethyl-3H-benzimidazole-5-sulfonic acid dimethylamide

A suspension of 0.5 g (1.86 mmol) 7-amino-2,3-dimethyl-3H-benzimidazole-5-sulfonic acid dimethylamide and 0.3 g (1.96 mmol) 2,6-dimethyl-benzyl chloride was stirred at room temperature and 0.08 g (2.05 mmol) sodium hydride (60% dispersion in mineral oil) were added. The reaction mixture was warmed to 45° C. and an additional amount of 0.3 g (1.96 mmol) 2,6-dimethyl-benzyl chloride and 0.13 g (3.25 mmol) sodium hydride were added in two portions over a period of 1.5 h. The mixture was stirred for 16 h at 40° C., cooled down and partitioned between dichloromethane and water. The organic layer was separated, dried over anhydrous magnesium sulphate and evaporated. Purification of the residue by column chromatography on silica gel using ethyl acetate:light petroleum ether (75:25) and crystallization from ethyl acetate/n-heptane yielded 0.35 g (49%) of the title compound as a colourless solid (m.p. 237-238° C.).

### 64. Ethyl1-benzyloxymethyl-4-(2,6-dimethyl-benzylamino)-2-methyl-1H-benzimidazole-6-carboxylate

To a solution of 6.5 g (14.2 mmol) 1-benzyloxymethyl-6-bromo-4-(2,6-dimethyl-benzylamino)-2-methyl-1H-benzimidazole in 125 ml ethanol and 25 ml triethylamine were added 1.0 g (1.36 mmol) bis(triphenylphosphine)palladium(II) chloride. The mixture was transferred to an autoclave and carbonylated (6 bar carbon monoxide pressure, 100° C.) for 16 h. The reaction mixture was cooled down and filtered. The filtrate was partitioned between dichloromethane and water. The organic layer was separated, dried over anhydrous magnesium sulphate and evaporated. Purification of the residue by column chromatography on silica gel using dichloromethane:methanol (100:1) and crystallization from ethyl acetate/n-heptane yielded 5.3 g (82%) of the title compound as a colourless solid (m.p. 155-156° C.).

### 65. 1-Benzyloxymethyl-4-(2,6-dimethyl-benzylamino)-2-methyl-1H-benzimidazole-6-carboxylic Acid

To a suspension of 5.0 g (10.9 mmol) ethyl 1-benzyloxymethyl-4-(2,6-dimethyl-benzylamino)-2-methyl-1H-benzimidazole-6-carboxylate in 30 ml dioxane were added 15 ml 2N aqueous sodium hydroxide. After 2 h at 90° C., the reaction mixture was cooled down and the pH was adjusted to pH=7 by adding 6N hydrochloric acid. After addition of 50 g silica gel, the mixture was evaporated to dryness and the residue was put on a column and eluted with dichloromethane:methanol (13:1). Evaporation of the solvent left a solid, which was crystallized from diethyl ether to give 4.36 g of the title compound which was used without further purification for the next step.

### 66. 1-Benzyloxymethyl-6-(N,N-dimethylaminocarbonyl)-4-(2,6-dimethyl-benzylamino)-2-methyl-1H-benzimidazole

To a suspension of 3.0 g (7 mmol, crude product) 1-benzyloxymethyl-4-(2,6-dimethyl-benzylamino)-2-methyl-1H-benzimidazole-6-carboxylic acid in 30 ml tetrahydrofuran and 15 ml N,N-dimethylformamide were added 1.97 g (12.2 mmol) N,N′-carbonyldiimidazole. After 2 h at 60° C., the solution was cooled down to 40° C. and 5.0 ml (25 mmol) dimethylamine (5M in tetrahydrofuran) were added. After 30 min, saturated aqueous ammonium chloride was added and the mixture was extracted with dichloromethane. The organic layer was separated, dried over anhydrous magnesium sulphate and evaporated. Purification of the residue by column chromatography on silica gel using ethyl acetate:light petroleum ether (75:25) yielded 2.68 g (84%) of the title compound as a colourless foam (m.p. 55-56° C.).

### 67. 6-(N,N-Dimethylaminocarbonyl)-4-(2,6-dimethyl-benzylamino)-2-methyl-1H-benzimidazole

A suspension of 2.4 g (7.1 mmol) 1-benzyloxymethyl-6-N,N-dimethylaminocarbonyl)-4-(2,6-dimethyl-benzylamino)-2-methyl-1H-benzimidazole, 4.5 g (71 mmol) ammonium formate and 0.5 g palladium on charcoal (10%) in 50 ml ethanol was heated to reflux. After 2 h, the mixture was filtered through celite and evaporated. The residue was partitioned between dichloromethane and water. The organic layer was separated, dried over anhydrous magnesium sulphate and evaporated. Purification of the residue by crystallization from ethyl acetate/n-heptane yielded 1.4 g (59%) of the title compound as a colourless solid (m.p. 203° C.).

### 68. 8-Benzyloxymethyl-6-(N-methylaminocarbonyl)-4-(2,6-dimethyl-benzylamino)-2-methyl-1H-benzimidazole

To a suspension of 1.3 g (3 mmol, crude product) 1-benzyloxymethyl-4-(2,6-dimethyl-benzylamino)-2-methyl-1H-benzimidazole-6-carboxylic acid in 20 ml tetrahydrofuran and 10 ml N,N-dimethylformamide were added 0.8 g (4.9 mmol) N,N′-carbonyldiimidazole. After 2.5 h at 60° C., the solution was cooled down to 40° C. and 5.0 ml (10 mmol) methylamine (2M in tetrahydrofuran) were added. After 1 h, saturated aqueous ammonium chloride was added and the mixture was extracted with dichloromethane. The organic layer was separated, dried over anhydrous magnesium sulphate and evaporated. Purification of the residue by crystallization from ethyl acetate/n-heptane yielded 0.91 g (71%) of the title compound as a colourless solid (m.p. 215-216° C.).

### 69. 4-(2,6-Dimethyl-benzylamino)-6-(N-methylaminocarbonyl)-2-methyl-1H-benzimidazole

A suspension of 0.85 g (1.92 mmol) 1-benzyloxymethyl-6-(N-methylaminocarbonyl)-4-(2,6-dimethyl-benzylamino)-2-methyl-1H-benzimidazole, 2.5 g (40 mmol) ammonium formate and 0.26 g palladium on charcoal (10%) in 45 ml ethanol was heated to reflux. After 1.5 h, the mixture was filtered through celite and evaporated. The residue was partitioned between dichloromethane and water. The organic layer was separated, dried over anhydrous magnesium sulphate and evaporated. Purification of the residue by crystallization from ethyl acetate/n-heptane yielded 0.47 g (76%) of the title compound as a colourless solid (m.p. 298° C.).

### 70. 6-(N,N-Dimethylaminocarbonyl)-4-(2-hydroxymethyl-6-methyl-benzylamino)-1,2-dimethyl-1H-benzimidazole

To a solution of 0.45 g (1.2 mmol) 6-bromo-4-(2-hydroxymethyl-6-methyl-benzylamino)-1,2-dimethyl-1H-benzimidazole in 5 ml dimethylamine and 15 ml tetrahydrofuran were added 0.125 g (0.18 mmol) bis(triphenylphosphine)palladium(II) chloride. The mixture was transferred to an autoclave and carbonylated (6 bar carbon monoxide pressure, 120° C.) for 16 h. The reaction mixture was cooled down and filtered. The filtrate was partitioned between dichloromethane and saturated aqueous ammonium chloride. The organic layer was separated, dried over anhydrous magnesium sulphate and evaporated. Purification of the residue by column chromatography on silica gel using dichloromethane:methanol (20:1) and crystallization from ethyl acetate/n-heptane yielded 0.07 g (15%) of the title compound as a colourless solid (m.p. 197-198° C.).

### 71. 6-Cyano-4-(2,6-dimethyl-benzylamino)-1,2-dimethyl-1H-benzimidazole

A suspension of 6.0 g (16.8 mmol) 6-bromo-4-(2,6-dimethyl-benzylamino)-1,2-dimethyl-1H-benzimidazole, 2.1 g (17.9 mmol) zinc cyanide and 1.94 g (1.68 mmol) tetrakis(triphenylphosphine)palladium in 50 ml degassed N,N-dimethylformamide was heated to 100° C. After 40 min, the reaction mixture was cooled down, poured into 300 ml saturated aqueous ammonium chloride and extracted with dichloromethane. The organic layer was separated, dried over anhydrous magnesium sulphate and evaporated. Purification of the residue by column chromatography on silica gel using dichloromethane:methanol (100:1) and crystallization from ethyl acetate yielded 4.56 g of the title compound as a colourless solid which was used without further purification for the next step.

### 72. 6-(4,5-Dihydro-1H-imidazol-2-yl)-4-(2,6-dimethyl-benzylamino)-1,2-dimethyl-1H-benzimidazole

A suspension of 0.5 g (1.64 mmol) 6-cyano-4-(2,6-dimethyl-benzylamino)-1,2-dimethyl-1H-benzimidazole and a catalytic amount of phosphorus pentasulfide in 5 ml ethylenediamine was heated to 120° C. After 1 h, the reaction mixture was cooled down and partitioned between water and dichloromethane. The organic layer was separated, dried over anhydrous magnesium sulphate and evaporated. Purification of the residue by crystallization from diethyl ether yielded 0.54 g (90%) of the title compound as a yellow solid (m.p. 273-274° C.).

### 73. 6-(4,5-Dihydro-1-methyl-1H-imidazol-2-yl)-4-(2,6-dimethyl-benzylamino)-1,2-dimethyl-1H-benzimidazole

A suspension of 0.25 g (0.82 mmol) 6-cyano-4-(2,6-dimethyl-benzylamino)-1,2-dimethyl-1H-benzimidazole and a catalytic amount of phosphorus pentasulfide in 2.5 ml N-methyl-ethylenediamine was heated to 120° C. After 1 h, the reaction mixture was cooled down and partitioned between water and dichloromethane. The organic layer was separated, dried over anhydrous magnesium sulphate and evaporated. Purification of the residue by column chromatography on silica gel using dichloromethane: methanol (4:1) and crystallization from diethyl ether yielded 0.11 g (37%) of the title compound as a colourless solid (m.p. 118-119° C.).

### 74. 6-(4,5-Dihydro-oxazol-2-yl)-4-(2,6-dimethyl-benzylamino)-1,2-dimethyl-1H-benzimidazole

A suspension of 0.5 g (1.64 mmol) 6-cyano-4-(2,6-dimethyl-benzylamino)-1,2-dimethyl-1H-benzimidazole and 0.54 g (3.9 mmol) zinc chloride in 5 ml ethanolamine was heated to 140° C. After 3.5 h, the reaction mixture was cooled down and partitioned between saturated aqueous ammonium chloride and dichloromethane. The organic layer was separated, dried over anhydrous magnesium sulphate and evaporated. Purification of the residue by column chromatography on silica gel using ethyl acetate and crystallization from ethyl acetate/diethyl ether yielded 0.25 g (42%) of the title compound as a colourless solid (m.p. 235° C.).

### 75. 4-2,6-Dimethyl-benzylamino)-N-hydroxy-1,2-dimethyl-1H-benzimidazole-6-carboxamidine

A suspension of 0.5 g (1.64 mmol) 6-cyano-4-2,6-dimethyl-benzylamino)-1,2-dimethyl-1H-benzimidazole, 0.67 g (9.6 mmol) hydroxylamine hydrochloride and 1.04 g (9.8 mmol) sodium carbonate in 10 ml N,N-dimethylformamide was heated to 110° C. After 7 h, the reaction mixture was cooled down and partitioned between saturated aqueous ammonium chloride and dichloromethane. The organic layer was separated, dried over anhydrous magnesium sulphate and evaporated. Purification of the residue by column chromatography on silica gel using dichloromethane:methanol (13:1) and crystallization from acetone yielded 0.39 g (71%) of the title compound as a colourless solid (m.p. 230° C.).

### 76. 6-Bromo-4-(2,6-dimethyl-benzylamino)-1-hydroxy-2-methyl-1H-benzimidazole

To a suspension of 4.0 g (16.5 mmol) 4-amino-6-bromo-1-hydroxy-2-methyl-1H-benzimidazole and 2.5 g (18.6 mmol) 2,6-dimethyl-benzaldehyde in 80 ml methanol were added 3.1 g (49.3 mmol) sodium cyanoborohydride in small portions and the pH was maintained at pH=3 by gradual addition of methanolic hydrogen chloride. After 3 h, the reaction mixture was hydrolyzed with saturated aqueous sodium hydrogen carbonate and partitioned between dichloromethane and water. The organic layer was separated, dried over anhydrous magnesium sulphate and evaporated. Purification of the residue by crystallization from acetone yielded 2.4 g (40%) of the title compound as a colourless solid (m.p. 277° C.).

### 77. 6-Bromo-4-(2,6-dimethyl-benzylamino)-1-methoxy-2-methyl-1H-benzimidazole

To a suspension of 0.36 g (1.0 mmol) 6-bromo-4-(2,6-dimethyl-benzylamino)-1-hydroxy-2-methyl-1H-benzimidazole and 0.3 g (2.2 mmol) potassium carbonate in 3 ml acetone were added 0.7 ml (1.12 mmol) of methyl iodide (1.6M in acetone). After 4 h, the reaction mixture was partitioned between dichloromethane and saturated aqueous ammonium chloride. The organic layer was separated, dried over anhydrous magnesium sulphate and evaporated. Purification of the residue by crystallization from diethyl ether/n-heptane yielded 0.2 g (53%) of the title compound as a colourless solid (m.p. 171-172° C.).

78. Ethyl 4-(2,6-dimethyl-benzylamino)-1-methoxy-2-methyl-1H-benzimidazole-6-carboxylate

To a solution of 1.5 g (4.0 mmol) 6-bromo-4-(2,6-dimethyl-benzylamino)-1-methoxy-2-methyl-1H-benzimidazole in 60 ml ethanol and 3.3 ml triethylamine were added 0.35 g (0.5 mmol) bis(triphenylphosphine)palladium(II) chloride. The mixture was transferred to an autoclave and carbonylated (6 bar carbon monoxide pressure, 120° C.) for 16 h. The reaction mixture was cooled down and filtered through celite. The filtrate was evaporated and partitioned between dichloromethane and water. The organic layer was separated, dried over anhydrous magnesium sulphate and evaporated. Purification of the residue by column chromatography on silica gel using ethyl acetate:light petroleum ether (1:1) yielded 0.44 g (29%) of the title compound as a colourless solid (m.p. 165-166° C.).

### 79. 4-(2,6-Dimethyl-benzylamino)-1-methoxy-2-methyl-1H-benzimidazole-6-carboxylic acid

To a suspension of 0.43 g (1.17 mmol) ethyl 4-(2,6-dimethyl-benzylamino)-1-methoxy-2-methyl-1H-benzimidazole-6-carboxylate in 10 ml dioxane were added 2 ml 2N aqueous sodium hydroxide. After 8 h at 80° C. and stirring overnight at ambient temperature, the reaction mixture was cooled down and the pH was adjusted to pH=7 by adding 2N hydrochloric acid. After addition of 25 g silica gel, the mixture was evaporated to dryness and the residue was put on a column and eluted with dichloromethane:methanol (13:1). Evaporation of the solvent left a solid, which was crystallized from acetone to give 0.3 g (75%) of the title compound as a colourless solid (m.p. 248-249° C.).

### 80. 6-(N,N-Dimethylamino-carbonyl)-4-(2,6-dimethyl-benzylamino)-1-methoxy-2-methyl-1H-benzimidazole

To a suspension of 0.28 g (0.8 mmol) 4-(2,6-dimethyl-benzylamino)-1-methoxy-2-methyl-1H-benzimidazole-6-carboxylic acid in 15 ml dichloromethane and 3.5 ml N,N-dimethylformamide were added 0.5 g (1.6 mmol) O-(1H-benzotriazol-1-yl)-N,N,N′,N′-tetramethyl-uronium tetrafluoroborate (TBTU). After 1 h at 40° C., 2.4 ml (4.8 mmol) dimethylamine (2M in tetrahydrofuran) were added at ambient temperature. After 30 min, the reaction mixture was partitioned between 2N aqueous sodium hydroxide and dichloromethane. The organic layer was separated, dried over anhydrous magnesium sulphate and evaporated. The residue was purified by crystallization from diethyl ether to yield 0.24 g (80%) of the title compound as a colourless solid (m.p. 128-129° C.).

### 81. 2-Acetoxymethyl-6-bromo-4-(2,6-dimethyl-benzylamino)-1-methyl-1H-benzimidazole

A suspension of 1.0 g (3.35 mmol) 2-acetoxymethyl-4-amino-6-bromo-1-methyl-1H-benzimidazole, 0.54 g (3.5 mmol) 2,6-dimethyl-benzyl chloride, 0.93 g (6.7 mmol) potassium carbonate and a catalytic amount of potassium iodide in 15 ml acetonitrile was heated to 70° C. After 4 h, a further amount of 0.27 g (1.75 mmol) 2,6-dimethyl-benzyl chloride were added and stirring was continued for 2 h. The reaction mixture was poured into 50 ml water and extracted with dichloromethane. The organic layer was separated, dried over anhydrous magnesium sulphate and evaporated. Purification of the residue by column chromatography on silica gel using ethyl acetate:light petroleum ether (7:3) and crystallization from ethyl acetate/n-heptane yielded 0.58 g (42%) of the title compound as a colourless solid (m.p. 188-190° C.).

### 82. 6-(N,N-Dimethylaminocarbonyl)-4-(2,6-dimethyl-benzylamino)-2-hydroxymethyl-1-methyl-1H-benzimidazole

To a solution of 2.0 g (4.8 mmol) 2-acetoxymethyl-6-bromo-4-(2,6-dimethyl-benzylamino)-1-methyl-1H-benzimidazole in 30 ml dimethylamine (2M in tetrahydrofuran) were added 0.67 g (0.95 mmol) bis(triphenylphosphine)palladium(II) chloride. The mixture was transferred to an autoclave and carbonylated (6 bar carbon monoxide pressure, 120° C.) for 25 h. The reaction mixture was cooled down and filtered. The filtrate was partitioned between saturated aqueous ammonium chloride and dichloromethane. The organic layer was separated, dried over anhydrous magnesium sulphate and evaporated. The residue was dissolved in 20 ml methanol and 0.1 g cesium carbonate were added. The mixture was refluxed for 30 min, cooled down and evaporated. Purification of the residue by column chromatography on silica gel using dichloromethane:methanol (13:1) and crystallization from ethyl acetate yielded 0.5 g (26%) of the title compound as a colourless solid (m.p. 171-172° C.).

### 83. 6-(N,N-Dimethylaminocarbonyl)-4-(2-ethyl-6-methyl-benzylamino)-2-hydroxymethyl-1-methyl-1H-benzimidazole

To a solution of 0.5 g (1.28 mmol) 6-bromo-4-(2-ethyl-6-methyl-benzylamino)-2-hydroxymethyl-1-methyl-1H-benzimidazole in 10 ml dimethylamine (2M in tetrahydrofuran) and 20 ml tetrahydrofuran were added 0.03 g (0.128 mmol) palladium(II) acetate and 0.2 g (0.77 mmol) triphenylphosphine. The mixture was transferred to an autoclave and carbonylated (6 bar carbon monoxide pressure, 120° C.) for 16 h. The reaction mixture was cooled down, poured into water and extracted with dichloromethane. The organic layer was dried over anhydrous magnesium sulphate and evaporated. Purification of the residue by column chromatography on silica gel using ethyl acetate and crystallization from ethyl acetate/diisopropyl ether yielded 0.28 g (58%) of the title compound as a colourless solid (m.p. 138-141° C.).

### 84. 4-(2,6-Dimethyl-benzylamino)-6-(6-ethyl-[1,3,4]oxadiazol-2yl)-1,2-dimethyl-1H-benzimidazole

0.1 g (0.3 mmol) 4-(2,6-dimethyl-benzylamino)-1,2-dimethyl-1H-benzimidazole-6-carboxylic acid hydrazide were suspended in 5 ml triethyl orthopropionate. After 1 h at 140° C., the reaction mixture was cooled down and partitioned between dichloromethane and water. The organic layer was separated, dried over anhydrous magnesium sulphate and evaporated. Purification of the residue by crystallization from n-heptane yielded 0.08 g (71%) of the title compound as a yellow solid (m.p. 246-247° C.).

### 85. 4-(2,6-Dimethyl-benzylamino)-6-(5-methyl-[1,3,4]oxadiazol-2yl)-1,2-dimethyl-1H-benzimidazole

0.5 g (1.48 mmol) 4-(2,6-dimethyl-benzylamino)-1,2-dimethyl-1H-benzimidazole-6-carboxylic acid hydrazide were suspended in 7 ml triethyl orthoacetate. After 2 h at 140° C., the reaction mixture was cooled down and partitioned between dichloromethane and water. The organic layer was separated, dried over anhydrous magnesium sulphate and evaporated. Purification of the residue by column chromatography on silica gel using dichloromethane:methanol (20:1) and crystallization from diethyl ether yielded 0.2 g (37%) of the title compound as a colourless solid (m.p. 256-257° C.).

### 86. Ethyl 4-benzyloxy-2-cyclopropyl-1-methyl-1H-benzimidazole-6-carboxylate

To a solution of 2.9 g (8.1 mmol) 4-benzyloxy-6-bromo-2-cyclopropyl-1-methyl-1H-benzimidazole in 100 ml ethanol and 7 ml triethylamine were added 0.6 g (0.86 mmol) bis(triphenylphosphine)palladium(II) chloride. The mixture was transferred to an autoclave and carbonylated (10 bar carbon monoxide pressure, 100° C.) for 18 h. The reaction mixture was cooled down, filtered and evaporated. The residue was dissolved in dichloromethane and extracted with water. The organic layer was separated, dried over anhydrous magnesium sulphate and evaporated. Purification of the residue by column chromatography on silica gel using toluene:dioxane (10:1) and crystallization from ethyl acetate/light petroleum ether yielded 2.33 g (83%) of the title compound as a colourless solid (m.p. 120° C.).

The following compounds can be obtained in an analogous manner using analogous reaction steps as described in the examples above:
a) 6-(N,N-Dimethylaminocarbonyl)-2-(N,N-dimethylamino)-4-(2,6-dimethyl-benzylamino)-1-methyl-1H-benzimidazole
b) 6-(N,N-Dimethylaminocarbonyl)-2-(N,N-dimethylamino)-4-(2-ethyl-6-methyl-benzylamino)-1-methyl-1H-benzimidazole
   Commercial Utility

The compounds of the formula 1 and 2 and their salts have valuable pharmacological properties which make them commercially utilizable. In particular, they exhibit marked inhibition of gastric acid secretion and an excellent gastric and intestinal protective action in warm-blooded animals, in particular humans. In this connection, the compounds according to the invention are distinguished by a high selectivity of action, an advantageous duration of action, a particularly good enteral activity, the absence of significant side effects and a large therapeutic range.

“Gastric and intestinal protection” in this connection is understood as meaning the prevention and treatment of gastrointestinal diseases, in particular of gastrointestinal inflammatory diseases and lesions (such as, for example, gastric ulcer, duodenal ulcer, gastritis, hyperacidic or medicament-related functional dyspepsia), which can be caused, for example, by microorganisms (e.g. Helicobacter pylori), bacterial toxins, medicaments (e.g. certain antiinflammatories and antirheumatics), chemicals (e.g. ethanol), gastric acid or stress situations.

In their excellent properties, the compounds according to the invention surprisingly prove to be clearly superior to the compounds known from the prior art in various models in which the antiulcerogenic and the antisecretory properties are determined. On account of these properties, the compounds of the formula 1 and 2 and their pharmacologically acceptable salts are outstandingly suitable for use in human and veterinary medicine, where they are used, in particular, for the treatment and/or prophylaxis of disorders of the stomach and/or intestine.

A further subject of the invention are therefore the compounds according to the invention for use in the treatment and/or prophylaxis of the abovementioned diseases.

The invention likewise includes the use of the compounds according to the invention for the production of medicaments which are employed for the treatment and/or prophylaxis of the abovementioned diseases.

The invention furthermore includes the use of the compounds according to the invention for the treatment and/or prophylaxis of the abovementioned diseases.

A further subject of the invention are medicaments which comprise one or more compounds of the formula 1 and/or their pharmacologically acceptable salts.

The medicaments are prepared by processes which are known per se and familiar to the person skilled in the art. As medicaments, the pharmacologically active compounds according to the invention (=active compounds) are either employed as such, or preferably in combination with suitable pharmaceutical auxiliaries or excipients in the form of tablets, coated tablets, capsules, suppositories, patches (e.g. as TTS), emulsions, suspensions or solutions, the active compound content advantageously being between 0.1 and 95% and it being possible to obtain a pharmaceutical administration form exactly adapted to the active compound and/or to the desired onset and/or duration of action (e.g. a sustained-release form or an enteric form) by means of the appropriate selection of the auxiliaries and excipients.

The auxiliaries and excipients which are suitable for the desired pharmaceutical formulations are known to the person skilled in the art on the basis of his/her expert knowledge. In addition to solvents, gel-forming agents, suppository bases, tablet auxiliaries and other active compound excipients, it is possible to use, for example, antioxidants, dispersants, emulsifiers, antifoams, flavor corrigents, preservatives, solubilizers, colorants or, in particular, permeation promoters and complexing agents (e.g. cyclodextrins).

The active compounds can be administered orally, parenterally or percutaneously.

In general, it has proven advantageous in human medicine to administer the active compound(s) in the case of oral administration in a daily dose of approximately 0.01 to approximately 20, preferably 0.05 to 5, in particular 0.1 to 1.5, mg/kg of body weight, if appropriate in the form of several, preferably 1 to 4, individual doses to achieve the desired result. In the case of a parenteral treatment, similar or (in particular in the case of the intravenous administration of the active compounds), as a rule, lower doses can be used. The establishment of the optimal dose and manner of administration of the active compounds necessary in each case can easily be carried out by any person skilled in the art on the basis of his/her expert knowledge.

If the compounds according to the invention and/or their salts are to be used for the treatment of the abovementioned diseases, the pharmaceutical preparations can also contain one or more pharmacologically active constituents of other groups of medicaments, for example: tranquillizers (for example from the group of the benzodiazepines, for example diazepam), spasmolytics (for example, bietamiverine or camylofine), anticholinergics (for example, oxyphencyclimine or phencarbamide), local anesthetics, (for example, tetracaine or procaine), and, if appropriate, also enzymes, vitamins or amino acids.

To be emphasized in this connection is in particular the combination of the compounds according to the invention with pharmaceuticals which inhibit acid secretion, such as, for example, H₂ blockers (e.g. cimetidine, ranitidine), H⁺/K⁺ ATPase inhibitors (e.g. omeprazole, pantoprazole), or further with so-called peripheral anticholinergics (e.g. pirenzepine, telenzepine) and with gastrin antagonists with the aim of increasing the principal action in an additive or super-additive sense and/or of eliminating or of decreasing the side effects, or further the combination with antibacterially active substances (such as, for example, cephalosporins, tetracyclines, penicillins, macrolides, nitroimidazoles or alternatively bismuth salts) for the control of Helicobacter pylori. Suitable antibacterial co-components which may be mentioned are, for example, mezlocillin, ampicillin, amoxicillin, cefalothin, cefoxitin, cefotaxime, imipenem, gentamycin, amikacin, erythromycin, ciprofloxacin, metronidazole, clarithromycin, azithromycin and combinations thereof (for example clarithromycin+metronidazole).

In view of their excellent gastric and intestinal protection action, the compounds of formula 1 are suited for a free or fixed combination with those medicaments (e.g. certain antiinflammatories and antirheumatics, such as NSAIDS), which are known to have a certain ulcerogenic potence.

Pharmacology

The excellent gastric protective action and the gastric acid secretion-inhibiting action of the compounds according to the invention can be demonstrated in investigations on animal experimental models. The compounds according to the invention investigated in the model mentioned below have been provided with numbers which correspond to the numbers of these compounds in the examples.

Testing of the Secretion-Inhibiting Action on the Perfused Rat Stomach

In Table A which follows, the influence of the compounds according to the invention on the pentagastrin-stimulated acid secretion of the perfused rat stomach after intraduodenal administration in vivo is shown.

TABLE A
Biological Data

| | Dose | Inhibition of |
|---|---|---|
| | (μmol/kg) | acid secretion |
| No. | i.d. | (%) |
| 8 | 1 | >50 |
| 10 | 1 | >50 |
| 11 | 1 | >50 |
| 13 | 1 | >50 |
| 19 | 1 | >50 |
| 35 | 1 | >50 |
| 38 | 1 | >50 |
| 40 | 1 | >50 |
| 41 | 1 | >50 |
| 42 | 1 | >50 |
| 43 | 1 | >50 |
| 44 | 1 | >50 |
| 45 | 1 | >50 |
| 46 | 1 | >50 |
| 49 | 1 | >50 |
| 55 | 1 | >50 |

Methodology

The abdomen of anesthetized rats (CD rat, female, 200-250 g; 1.5 g/kg i.m. urethane) was opened after tracheotomy by a median upper abdominal incision and a PVC catheter was fixed transorally in the esophagus and another via the pylorus such that the ends of the tubes just projected into the gastric lumen. The catheter leading form the pylorus led outward into the right abdominal wall through a side opening.

After thorough rinsing (about 50-100 ml), warm (37° C.) physiological NaCl solution was continuously passed through the stomach (0.5 ml/min, pH 6.8-6.9; Braun-Unita I). The pH (pH meter 632, glass electrode EA 147; φ=5 mm, Metrohm) and, by titration with a freshly prepared 0.01 N NaOH solution to pH 7 (Dosimat 665 Metrohm), the secreted HCl were determined in the effluent in each case collected at an interval of 15 minutes.

The gastric secretion was stimulated by continuous infusion of 1 μg/kg (=1.65 ml/h) of i.v. pentagastrin (left femoral vein) about 30 min after the end of the operation (i.e. after determination of 2 preliminary fractions). The substances to be tested were administered intraduodenally in a 2.5 ml/kg liquid volume 60 min after the start of the continuous pentagastrin infusion.

The body temperature of the animals was kept at a constant 37.8-38° C. by infrared irradiation and heat pads (automatic, stepless control by means of a rectal temperature sensor).

1. A compound of the formula 1 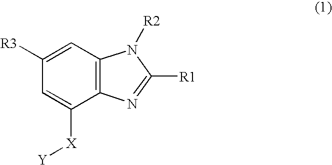
in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl, mono- or di-1-4C-alkylamino or 1-4C-alkylcarbonyloxy-1-4C-alkyl,
R2 is hydrogen, 1-4C-alkyl, aryl, 3-7C-cycloalkyl, 3-7C-cycloalkyl-1-4C-alkyl, 1-4C-alkoxycarbonyl, mono or di-1-4C-alkylamino-1-4C-alkylcarbonyl, hydroxy-1-4C-alkyl, fluoro-2-4C-alkyl, aryl-1-4C-alkoxy-1-4C-alkyl, hydroxy or 1-4C-alkoxy,
R3 is hydrogen, halogen, fluoro-1-4C-alkyl, carboxyl, —CO-1-4C-alkoxy, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, cyano, the group —CO—NR31R32, the group SO₂—NR31R32 or the group Het,
where
R31 is hydrogen, hydroxyl, 1-7C-alkyl, hydroxy-1-4C-alkyl, 1-4C-alkoxy-1-4C-alkyl or 3-7C-cycloalkyl, amino and
R32 is hydrogen, 1-7C-alkyl, hydroxy-1-4C-alkyl or 1-4C-alkoxy-1-4C-alkyl,
or where
R31 and R32 together, including the nitrogen atom to which both are bonded, are a pyrrolidino, piperidino, piperazino, N-1-4C-alkylpiperazino, morpholino, aziridino or azetidino group and
Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol and tetrazol,
where
R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R34 is hydrogen, 1-4C-alkyl, 1-4C-alkoxy, 1-4C-alkoxycarbonyl, halogen, trifluoromethyl or hydroxy,
R35 is hydrogen, 1-4C-alkyl, 1-4C-alkoxy, 1-4C-alkoxycarbonyl, halogen, trifluoromethyl or hydroxy,
X is O (oxygen) or NH and
Y has either the meaning —CH₂—Ar
wherein
Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl, or Y denotes the group gp 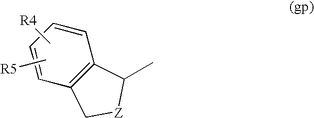
wherein
Z has the meaning —CHR8- or —CHR8-CHR9-
wherein Ar and/or in the group gp
R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, 1-4C-alkoxy, 1-4C-alkoxycarbonyl, halogen, trifluoromethyl or hydroxy,
R6 is hydrogen, 1-4C-alkyl or halogen and
R7 is hydrogen, 1-4C-alkyl or halogen,
R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy,
R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy,
and wherein
aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
with the proviso that R3 does not have the meaning hydrogen or halogen when Y denotes —CH₂—Ar and R2 denotes hydrogen, 1-4C-alkyl or 3-7C-cycloalkyl-1-4C-alkyl,
or a hydrate, solvate, salt, hydate of a salt or solvate of a salt thereof.

2. A compound as claimed in claim 1,
in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is hydrogen, 1-4C-alkyl, aryl, 3-7C-cycloalkyl, 3-7C-cycloalkyl-1-4C-alkyl, 1-4C-alkoxycarbonyl, mono or di-1-4C-alkylamino-1-4C-alkylcarbonyl, hydroxy-1-4C-alkyl or fluoro-2-4C-alkyl,
R3 is hydrogen, halogen, fluoro-1-4C-alkyl, carboxyl, —CO-1-4C-alkoxy, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the group —CO—NR31R32,
where
R31 is hydrogen, hydroxyl, 1-7C-alkyl, hydroxy-1-4C-alkyl or 1-4C-alkoxy-1-4C-alkyl
R32 is hydrogen, 1-7C-alkyl, hydroxy-1-4C-alkyl or 1-4C-alkoxy-1-4C-alkyl,
or where
R31 and R32 together, including the nitrogen atom to which both are bonded, are a pyrrolidino, piperidino, piperazino, N-1-4C-alkylpiperazino or morpholino group,
X is O (oxygen) or NH and
Y has either the meaning —CH₂—Ar
wherein
Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
or Y denotes the group gp 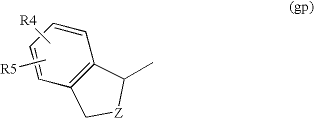
wherein
Z has the meaning —CHR8- or —CHR8-CHR9-
wherein Ar and/or in the group gp
R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, 1-4C-alkoxy, 1-4C-alkoxycarbonyl, halogen, trifluoromethyl or hydroxy,
R6 is hydrogen, 1-4C-alkyl or halogen and
R7 is hydrogen, 1-4C-alkyl or halogen,
R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy or 1-4C-alkoxy-1-4C-alkoxycarbonylamino,
and wherein
aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
with the proviso that R3 does not have the meaning hydrogen or halogen when Y denotes —CH₂—Ar,
or a hydrate, solvate, salt, hydate of a salt or solvate of a salt thereof.

3. A compound as claimed in claim 1, characterized by the formula 1a 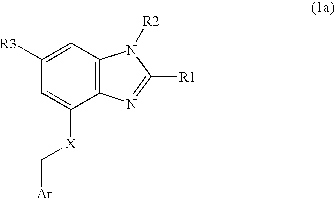
in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl, mono- or di-1-4C-alkylamino or 1-4C-alkylcarbonyloxy-1-4C-alkyl,
R2 is hydrogen, 1-4C-alkyl, aryl, 3-7C-cycloalkyl, 3-7C-cycloalkyl-1-4C-alkyl, 1-4C-alkoxycarbonyl, mono or di-1-4C-alkylamino-1-4C-alkylcarbonyl, hydroxy-1-4C-alkyl, fluoro-2-4C-alkyl, aryl-1-4C-alkoxy-1-4C-alkyl, hydroxy or 1-4C-alkoxy,
R3 is hydrogen, halogen, fluoro-1-4C-alkyl, carboxyl, —CO-1-4C-alkoxy, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, cyano, the group —CO—NR31R32, the group SO₂—NR31R32 or the group Het,
where
R31 is hydrogen, hydroxyl, 1-7C-alkyl, hydroxy-1-4C-alkyl, 1-4C-alkoxy-1-4C-alkyl or 3-7C-cycloalkyl, amino and
R32 is hydrogen, 1-7C-alkyl, hydroxy-1-4C-alkyl or 1-4C-alkoxy-1-4C-alkyl,
or where
R31 and R32 together, including the nitrogen atom to which both are bonded, are a pyrrolidino, piperidino, piperazino, N-1-4C-alkylpiperazino, morpholino, aziridino or azetidino group and
Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol and tetrazol,
where
R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R34 is hydrogen, 1-4C-alkyl, 1-4C-alkoxy, 1-4C-alkoxycarbonyl, halogen, trifluoromethyl or hydroxy,
R35 is hydrogen, 1-4C-alkyl, 1-4C-alkoxy, 1-4C-alkoxycarbonyl, halogen, trifluoromethyl or hydroxy,
X is O (oxygen) or NH and
Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
where
R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, 1-4C-alkoxy, 1-4C-alkoxycarbonyl, halogen, trifluoromethyl or hydroxy,
R6 is hydrogen, 1-4C-alkyl or halogen and
R7 is hydrogen, 1-4C-alkyl or halogen,
and wherein
aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
with the proviso that R3 does not have the meaning hydrogen or halogen when Y denotes —CH₂—Ar and R2 denotes hydrogen, 1-4C-alkyl or 3-7C-cycloalkyl-1-4C-alkyl,
or a hydrate, solvate, salt, hydate of a salt or solvate of a salt thereof.

4. A compound as claimed in claim 1, characterized by the formula 1b 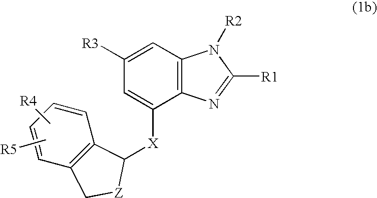
in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl, mono- or di-1-4C-alkylamino or 1-4C-alkylcarbonyloxy-1-4C-alkyl,
R2 is hydrogen, 1-4C-alkyl, aryl, 3-7C-cycloalkyl, 3-7C-cycloalkyl-1-4C-alkyl, 1-4C-alkoxycarbonyl, mono or di-1-4C-alkylamino-1-4C-alkylcarbonyl, hydroxy-1-4C-alkyl, fluoro-2-4C-alkyl, aryl-1-4C-alkoxy-1-4C-alkyl, hydroxy or 1-4C-alkoxy,
R3 is hydrogen, halogen, fluoro-1-4C-alkyl, carboxyl, —CO-1-4C-alkoxy, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, cyano, the group —CO—NR31R32, the group SO₂—NR31R32 or the group Het,
where
R31 is hydrogen, hydroxyl, 1-7C-alkyl, hydroxy-1-4C-alkyl, 1-4C-alkoxy-1-4C-alkyl or 3-7C-cycloalkyl, amino and
R32 is hydrogen, 1-7C-alkyl, hydroxy-1-4C-alkyl or 1-4C-alkoxy-1-4C-alkyl,
or where
R31 and R32 together, including the nitrogen atom to which both are bonded, are a pyrrolidino, piperidino, piperazino, N-1-4C-alkylpiperazino, morpholino, aziridino or azetidino group and
Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol and tetrazol,
where
R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R34 is hydrogen, 1-4C-alkyl, 1-4C-alkoxy, 1-4C-alkoxycarbonyl, halogen, trifluoromethyl or hydroxy,
R35 is hydrogen, 1-4C-alkyl, 1-4C-alkoxy, 1-4C-alkoxycarbonyl, halogen, trifluoromethyl or hydroxy,
R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, 1-4C-alkoxy, 1-4C-alkoxycarbonyl, halogen, trifluoromethyl or hydroxy,
X is O (oxygen) or NH and
Z has the meaning —CHR8- or —CHR8-CHR9-
where
R8 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy,
R9 is hydrogen, 1-7C-alkyl, 2-7C-alkenyl, hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy,
and wherein
aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
or a hydrate, solvate, salt, hydate of a salt or solvate of a salt thereof.

5. A compound of formula 1a as claimed in claim 3, characterized by the formula 1a-1 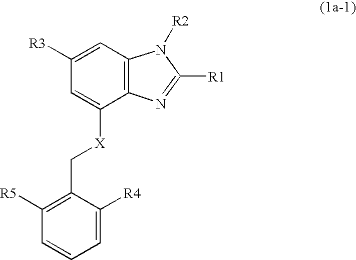
where
R1 is hydrogen, 1-4C-alkyl, 3-7C-cycloalkyl, hydroxy-1-4C-alkyl or 1-4C-alkoxy-1-4C-alkyl,
R2 is hydrogen, 1-4C-alkyl, hydroxy, 1-4C-alkoxy or aryl-1-4C-alkoxy-1-4C-alkyl,
R3 is carboxyl, —CO-1-4C-alkoxy, hydroxy-1-4C-alkyl, 1-4C-alkoxy-1-4C-alkyl, cyano, the group —CO—NR31R32, the group SO₂—NR31R32 or the group Het,
where
R31 is hydrogen, 1-7C-alkyl, hydroxy-1-4C-alkyl, 1-4C-alkoxy-1-4C-alkyl, 3-7C-cycloalkyl or amino and
R32 is hydrogen or 1-7C-alkyl,
or where
R31 and R32 together, including the nitrogen atom to which both are bonded, are a pyrrolidino, piperidino, piperazino, N-1-4C-alkylpiperazino, morpholino, aziridino or azetidino group and
Het is a heterocyclic residue, substituted by R33, R34 and R35, selected from the group consisting of oxadiazol, dihydrooxazol and dihydroimidazol,
where
R33 is hydrogen or 1-4C-alkyl,
R34 is hydrogen or 1-4C-alkyl,
R35 is hydrogen or 1-4C-alkyl,
R4 is hydrogen, 1-4C-alkyl, hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
R5 is hydrogen, 1-4C-alkyl or 1-4C-alkoxy and
X is O (oxygen) or NH,
or a hydrate, solvate, salt, hydate of a salt or solvate of a salt thereof.

6. A compound of formula 1b as claimed in claim 4, characterized by the formula 1b-1 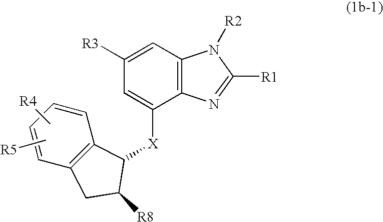
in which
R1 is 1-4C-alkyl or 3-7C-cycloalkyl,
R2 is hydrogen or 1-4C-alkyl,
R3 is carboxyl, —CO-1-4C-alkoxy, 1-4C-alkoxy-1-4C-alkyl, 1-4C-alkoxy-1-4C-alkoxy-1-4C-alkyl or the group —CO—NR31R32,
where
R31 is hydrogen, 1-7C-alkyl, hydroxy-1-4C-alkyl, 1-4C-alkoxy-1-4C-alkyl or 3-7C-cycloalkyl and
R32 is hydrogen, 1-7C-alkyl, hydroxy-1-4C-alkyl or 1-4C-alkoxy-1-4C-alkyl,
or where
R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, 1-4C-alkoxy or halogen,
R5 is hydrogen or 1-4C-alkyl,
R8 is hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy,
X is O (oxygen) or NH,
or a hydrate, solvate, salt, hydate of a salt or solvate of a salt thereof.

7. A compound of the formula 1b-1 as claimed in claim 6,
in which
R1 is 1-4C-alkyl,
R2 is hydrogen or 1-4C-alkyl,
R3 is carboxyl, —CO-1-4C-alkoxy, 1-4C-alkoxy-1-4C-alkyl, 1-4C-alkoxy-1-4C-alkoxy-1-4C-alkyl or the group —CO—NR31R32,
where
R31 is hydrogen, 1-7C-alkyl, hydroxy-1-4C-alkyl, 1-4C-alkoxy-1-4C-alkyl or 3-7C-cycloalkyl and
R32 is hydrogen, 1-7C-alkyl, hydroxy-1-4C-alkyl or 1-4C-alkoxy-1-4C-alkyl,
or where
R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, 1-4C-alkoxy or halogen,
R5 is hydrogen,
R8 is hydroxyl, 1-4C-alkoxy, oxo-substituted 1-4C-alkoxy, 3-7C-cycloalkoxy, 3-7C-cycloalkyl-1-4C-alkoxy, hydroxy-1-4C-alkoxy, 1-4C-alkoxy-1-4C-alkoxy, 1-4C-alkoxy-1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, halo-1-4C-alkoxy, amino, mono- or di-1-4C-alkylamino, 1-4C-alkylcarbonylamino, 1-4C-alkoxycarbonylamino, mono- or di-1-4C-alkylamino-1-4C-alkylcarbonyloxy, 1-4C-alkoxy-1-4C-alkoxycarbonylamino or 1-4C-alkoxy-1-4C-alkylcarbonyloxy,
X is O (oxygen) or NH,
or a hydrate, solvate, salt, hydate of a salt or solvate of a salt thereof.

8. A compound of the formula 1a-1 as claimed in claim 5, in which
R1 is 1-4C-alkyl,
R2 is 1-4C-alkyl,
R3 is carboxyl, —CO-1-4C-alkoxy, 1-4C-alkoxy-1-4C-alkyl or the group —CO—NR31R32,
where
R31 is hydrogen, 1-4C-alkyl, hydroxy-1-4C-alkyl, 1-4C-alkoxy-1-4C-alkyl or 3-7C-cycloalkyl and
R32 is hydrogen or 1-4C-alkyl,
R4 is 1-4C-alkyl or 1-4C-alkylcarbonylamino,
R5 is 1-4C-alkyl,
X is O (oxygen) or NH,
or a hydrate, solvate, salt, hydate of a salt or solvate of a salt thereof.

9. The compound 6-(N,N-Dimethylaminocarbonyl)-4-(2-ethyl-6-methyl-benzylamino)-1,2-dimethyl-1H-benzimidazole, or a hydrate, solvate, salt, hydrate of a salt or solvate of a salt thereof.

10. The compound 6-(N,N-Dimethylaminocarbonyl)-4-(2,6-dimethyl-benzylamino)-1,2-dimethyl-1H-benzimidazole, or a hydrate, solvate, salt, hydate of a salt or solvate of a salt thereof.

11. A pharmaceutical composition comprising a compound as claimed in claim 1 and/or a pharmacologically acceptable hydrate, solvate, salt, hydate of a salt or solvate of a salt thereof, together with a suitable pharmaceutical auxiliary and/or excipient.

12. (canceled)

13. A method of treating or preventing a gastrointestinal disorder in a patient comprising administering to a patient in need thereof a therapeutically effective amount of a compound of formula 1 as claimed in claim 1, or a hydrate, solvate, salt, hydrate of a salt or solvate of a salt thereof.
